(19) United States
(12) Patent Application Publication
(10) Pub. No.: US 2003/0092037 A1
(43) Pub. Date: May 15, 2003

(54) Elk1 phosphorylation related gene

(76) Inventors: Osamu Matsuzaki, Shizuoka (JP); Akio Matsuda, Shizuoka (JP); Yukiko Nagano, Shizuoka (JP); Naomi Suzuki, Shizuoka (JP)

Correspondence Address: BIRCH STEWART KOLASCH & BIRCH, PO BOX 747, FALLS CHURCH, VA, 22040-0747, US

(21) Appl. No.: 10/197,666

(22) Filed: Jul. 18, 2002

Related U.S. Application Data
(60) Provisional application No. 60/305,884, filed on Jul. 18, 2001.
(60) Provisional application No. 60/316,304, filed on Sep. 4, 2001.
(60) Provisional application No. 60/350,027, filed on Jan. 23, 2002.

(30) Foreign Application Priority Data
Jul. 18, 2001 (JP) 218204/2001
Aug. 31, 2001 (JP) 263450/2001
Jan. 21, 2002 (JP) 012176/2002

(51) Int. Cl. C12Q 1/68; C07H 21/04; C12N 9/12; C12N 9/16; C12P 21/02; C12N 5/06

(52) U.S. Cl. 435/6; 435/194; 435/196; 435/69.1; 435/320.1; 435/325; 536/23.2

(57) ABSTRACT

Proteins having MAP kinase cascade activity, which are used for diagnosing, treating or preventing diseases associated with the excessive activation or inhibition of MAP kinase cascade are provided. Using the plasmids pFR-Luc and pFA2-Elk1, the cDNA encoding a protein capable of activating MAP kinase cascade was cloned from the cDNA library constructed from human lung fibroblasts, and the DNA sequence and the deduced amino acid sequence were determined. The protein, the DNA encoding the protein, a recombinant vector containing the DNA, and a transformant containing the recombinant vector are useful for screening a substance inhibiting or promoting activation of MAP kinase cascade.

### TECHNICAL FIELD

The present invention relates to a protein capable of phosphorylating Elk1 and/or activating a kinase which phosphorylates Elk1, a DNA sequence encoding the protein, a recombinant vector containing the DNA, a transformant containing the recombinant vector, and an antibody which reacts with the protein. The present invention also relates to use of the protein, DNA molecule or antibody of the invention in the diagnosis, treatment or prevention of diseases associated with the excessive activation or inhibition of Elk1 phosphorylation action or a kinase which phosphorylates Elk1.

The present invention also relates to a method for screening a substance capable of inhibiting or activating Elk1 phosphorylation action and/or a kinase which phosphorylates Elk1 by using the protein, DNA, recombinant vector and transformant.

### BACKGROUND ART

MAP (mitogen-activated protein) kinase (MAPK) was first identified in the second half of the 1980s as a serine/threonine kinase which is activated with stimulation of insulin (Sturgill, T. W. et al., Biochim. Biophys. Acta 1092: 350-357 (1991)), various cell growth factors and tumor promoter (Nishida, E. et al., Int. Rev. Cytol. 138: 211-238 (1992)). Studies over the past 10 years revealed that MAP kinase was a principal constituent in intracellular signal transduction involved in determining the destiny and controlling the function of eukaryotes in response to extracellular stimuli. (Nishida, B et al., Trends Biochem. Sci., 18: 128-131 (1993), Marshall, C. J., Curr. Opin. Genet. Dev., 4: 82-89 (1994), Cobb. M. H. et al., J. Biol. Chem., 270: 14843-14846 (1995)). In particular, the elucidation of the signal transduction pathway which starts from cell growth factor receptors having tyrosine kinase activity, through adaptor molecules composed of SH2 (src homology 2) and SH3 (src homology 3), Ras (an oncogene product which is a GTP-binding protein), and Raf-1 (an oncogene product which is serine/threonine kinase) and leading to MAP kinase, and the understanding that this signal transduction pathway is central pathway for determining cell proliferation, differentiation and development of higher eukaryotic organisms, have been great achievements in the field of cell biology in the 1990s.

For signal transduction molecules, the most important point is the switching on and off of its activity. In this respect, MAP kinase possesses unique and interesting properties. That is, for the activation of MAP kinase, phosphorylation of T and Y within Thr-Glu-Tyr (TEY) sequence between kinase sub-domain VII and VIII is required. Identified as an enzyme for the phosphorylation of both these amino acid residues, is MAPK kinase (MAPKK) or MAPK/ERK kinase (MEK). MAPKK is therefore a dual-specificity kinase which can phosphorylate serine/threonine and tyrosine. Further, MAPKK, for its activation, requires phosphorylation of two serine (or threonine) residues between kinase domains VII and VIII. The serine/threonine kinase responsible for this phosphorylation is called MAPKK kinase (MAPKKK). Raf-1 is one example of MAPKKK, and the Ras→Raf-1 (=MAPKKK)→MAPKK→MAPK chain is one of the major signal transduction pathways. The cascade reaction consisting of three molecules, MAPKKK/MAPKK/MAPK is called the MAP kinase cascade.

Since the above-described Raf-1(MAPKKK)-MAPKK-MAP kinase signal transduction system was first identified in the MAP kinase cascade, it is frequently referred to as the classical MAP kinase pathway. Thereafter, it became clear that MAP kinase-like kinases exist. One example of such a kinase is a Stress-Activated Protein Kinase (SAPK). This enzyme was identified as a kinase which is activated when a cell is subjected to chemical stress such as protein synthesis inhibiting agents or physical stress such as heat shock or osmotic pressure changes (Kyriakis, J. M., Nature, 369: 156-160 (1994)). SAPK was later found to be identical to c-Jun N-terminal Kinase (JNK), which increases transcription activity by phosphorylating the N-terminus of transcription factor Jun, and was independently identified at the same time. (Derijard, B., Cell, 76: 1025-1037 (1994)). SAPK/JNK has homology to MAP kinase, but the TEY sequence necessary for activation in classical MAP kinase is TPY (Thr-Pro-Tyr). The mechanism whereby activation is effected by the Thr and Tyr of this sequence being phosphorylated by a single upstream kinase MAPKK is similar to that of classical MAP kinase. Today, the following two kinases are known as MAPKKs for SAPK/JNK. One type is SAPK/ERK kinase-1 (SEK1) or mitogen-activated protein kinase kinase 4 (MKK4) (Lin, A., et al., Science, 268: 286-290 (1995), Sanchez, I., Nature, 372: 794-798 (1994), Moriguchi, T., et al., J. Biol. Chem., 270: 12969-12972 (1995)). The other type is mitogen activated protein kinase kinase 7 (MKK7) (Moriguchi, T., et al, EMBO J., 16: 7045-7053 (1997), Tournier, C., et al, P.N.A.S., 94: 7337-7342 (1997), and others). That is, MAPKK operates as an upstream factor against classical MAP kinase, and novel MAP kinase is phosphorylated and activated specifically by another MAPKK. As an upstream MAPKKK on the pathway leading to SAPK/JNK, MEKK1 is known, but it is anticipated that there exist other kinases that operate as MAPKKK.

There is one other kinase analogous to MAP kinase which is simply called p38 based on its molecular weight. This kinase was identified and cloned as a protein which is subject to tyrosine phosphorylation in an early stage after stimulating a lymphocyte (Han, J., et al., Science, 265: 808-811(1994)). At around the same time, CSAID binding protein (CSBP) was identified as protein which binds to cytokine-suppressive anti-inflammatory drug (CSAID); a drug suppressing the production of inflammatory cytokines in lymphocytes (Lee, J. C., et al., Nature, 372: 739-746 (1994)). At present, this has been clarified to be a human form of p38. Further, this is also the same molecule as MPK2 which was independently isolated to be a kinase which is activated by stress stimulation. In p38/CSBP/MPK2, the above-mentioned TXY (X is a predetermined amino acid residue) sequence is TGY, and p38/CSBP/MPK2 is activated by phosphorylation of the Thr and Tyr by a single upstream kinase, MAPKK. As MAPKKs which activate p38, MKK3 and MKK6 operate specifically (Moriguchi, T., et al., J. Biol. Chem., 271: 26981-26988 (1996), Cuenda, A., et al,. EMBO J., 15: 4156-4164 (1996)).

There is mutual homology between the sequences of classical MAP kinase and SAPK/JNK, p38/CSBP/MPK2 and they constitute a superfamily. Similarly, the MAPKKs specific to each of these three also have mutual homology, and MKK1, MKK2, MKK3, MKK4, MKK6, MKK7, etc belong to this superfamily. (Kyriakis, J. M., et al., J. Biol. Chem., 271: 24313-24316 (1996), Davis, R. J., Trends Biochem. Sci., 19: 470-473 (1994)). In contrast, between further upstream MAPKKKs which phosphorylate and thereby activate each MAPKK, mutual sequence homology is relatively low. There is only about 30% homology even in the kinase domain between kinases having MAPKKK activity such as Raf, TAK1, MEKK, MLK3, Ask1, Mos, Cot, etc. This matches well with the adaptedness for purpose of a system which specifically activates a necessary MAP kinase signal transmission pathway in response to a great variety of stresses.

SAPK/JNK and p38/CSBP/MPK2 are not activated by growth factors which activate classical MAP kinase, and are each known to be activated by stresses such as osmotic shock or heat shock, or by inflammatory cytokines such as TNF-α or IL-1, etc. (Kyriakis., J. M., et al., J. Biol. Chem., 271: 24313-24316 (1996), Davis., R. J., Trends Biochem. Sci., 19: 470-473 (1994)) Further, they are activated under a condition which induces cell death, such as UV radiation and depletion of serum and/or growth factor(Kyriakis., J. M., et al., J. Biol. Chem., 271: 24313-24316 (1996), Davis., R. J., Trends Biochem. Sci., 19: 470-473 (1994)). Unlike classical MAP kinase which is activated by a signal induced from a tyrosine kinase-type receptor, the systems of SAPK/JNK and p38/CSBP/MPK2 are characterized by the extreme variety of entry points thereto.

As described above, it is known that MAP kinase cascades, particularly the SAPK/JNK and p38/CSBP/MPK2 systems, are activated by a variety of stimuli. From among these, we focused on the immediate activation of SAPK/JNK and p38/CSBP/MPK2 after stimulation by extracellular inflammatory cytokines. In other words, the MAP kinase cascade is predicted to be deeply involved in inflammation response in vivo. Along the pathway from an extracellular stimulus finally to the activation of SAPK/JNK or p38/CSBP/MPK2, there exist many MAPKK kinases and MAPK kinases, or groups of adaptor molecular which connect these, and these have not yet been fully clarified.

### DISCLOSURE OF THE INVENTION

The object of the present invention is to identify a new gene and protein such as described above acting to activate MAP kinase cascade, and to provide a method of use of them in medicaments, diagnostics and therapy. Kinases such as p38, JNK, and ERK which are furthermost downstream in the MAP kinase cascade, often phosphorylate Elk1 as a substrate. Therefore, a group of proteins which act to activate MAP kinase cascade were selected using ELK1 phosphorylation action and/or activation of a kinase which phosphorylates ELK1 as an indicator.

That is, the present invention provides a new protein capable of phosphorylating Elk1 and/or activating kinase which phosphorylates Elk1, a DNA sequence encoding the protein, a recombinant vector containing the DNA, a transformant containing the recombinant vector, a process for producing the protein, an antibody directed against the protein or a peptide fragment thereof, and a process for producing the antibody.

The present invention also provides a method for screening a substance capable of inhibiting or activating Elk1 phosphorylation action and/or kinase which phosphorylates Elk1, a kit for the screening, a substance capable of inhibiting or activating Elk1 phosphorylation action and/or kinase which phosphorylates Elk1 obtainable by the screening method or the screening kit, a process for producing the substance, a pharmaceutical composition containing a substance capable of inhibiting or activating Elk1 phosphorylation action and/or kinase which phosphorylates Elk1, etc.

Recently, random analysis of cDNA molecules has been intensively carried out to analyze various genes, which are expressed in vivo. The cDNA fragments thus obtained have been entered for databases and published as ESTs (Expressed Sequence Tags, e.g., http//www.ncbi.nlm.nih.gov/dbEST). However, ESTs are merely sequence information, and it is difficult to predict their functions. ESTs are also arranged in UniGene (http//www.ncbi.nlm.nih.gov/UniGene), and about 95,000 human ESTs have been registered until now. However, most of these ESTs have their 5′ end nucleotide sequences deleted, and contain no translation initiation site. Therefore it is unlikely that such analysis will directly lead to gene functional analysis such as the analysis of protein functions on the assumption of the determination of mRNA coding regions and the understanding of gene expression control by the analysis of promoters.

On the other hand, one method to elucidate functions of gene products (i.e., proteins) is transient expression cloning method using animal cells [see e.g., “Idenshi Kougaku Handbook (Genetic Engineering Handbook)”, an extra issue of “Jikken Igaku (Experimental Medicine)”, YODOSHA CO., LTD.]. This method involves transfecting animal cells with a cDNA library constructed using an animal cell expression vector to directly express a functional protein, and identifying and cloning the cDNA based on the biological activity of the protein having an effect on the cells. This method requires no chemical information (amino acid sequences and molecular weights) regarding the target protein product as a prerequisite, and allows the identification of cDNA clones by detecting specific biological activity of the protein expressed in the cells or culture.

For the efficient expression cloning, there is a need to devise a method of preparing a cDNA library. Several methods have been widely used to construct cDNA libraries [e.g., the method of Gubbler-Hoffman: Gene 25 (1983); and the method of Okayama-Berg: Mol. Cell. Biol. 2 (1982)]. However, most of the cDNA molecules prepared by these methods have their 5′ end nucleotide sequences deleted, and thus these methods rarely produce full-length cDNA, a complete DNA copy of mRNA. This is because the reverse transcriptase used to prepare cDNA from mRNA does not necessarily have sufficiently high efficiency to enable production of full-length cDNA. Therefore it is necessary to improve these prior art methods in order to efficiently carry out the above expression cloning.

In addition, in order to carry out the functional analysis of genes, it is essential to clone full-length cDNA sequences and express proteins having their full length amino acid sequence. Therefore, it has been necessary to construct cDNA libraries containing enriched full-length cDNA for efficient expression cloning.

The present inventors have intensively studied to solve the above problems. As a result, the present inventors have succeeded in constructing a full-length cDNA library by using the oligo-capping method; establishing a gene function assay system employing an expression cloning method using HEK293EBNA cells; and isolating a new DNA (cDNA) encoding a protein having a function of phosphorylating Elk1 and/or activating kinase which phosphorylates Elk1 by using the assay system. This new DNA molecule induced Elk1 phosphorylation action or kinase which phosphorylates Elk1 by its expression in HEK293EBNA cells. This result shows that this new DNA is a signal transduction molecule involved in Elk1 phosphorylation action and/or kinase which phosphorylates Elk1. Thus, the present invention has been completed.

That is, the present invention relates to:

(1) A purified protein selected from the group consisting of:

(a) a protein which consists of an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 and 154; and

(b) a protein that acts to phosphorylate Elk1 and/or activate kinase which phosphorylates Elk1 and consists of an amino acid sequence having at least one amino acid deletion, substitution or addition in an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 and 154.

(2) A purified protein that acts to phosphorylate Elk1 and/or activate kinase which phosphorylates Elk1 and comprises an amino acid sequence having at least 95% identity to the protein according to above item (1) over the entire length thereof.

The proteins of (1) and (2) are preferably purified and isolated proteins.

(3) An isolated polynucleotide which comprises a nucleotide sequence encoding a protein selected from the group consisting of:

(a) a protein which comprises an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 and 154; and

(b) a protein that acts to phosphorylate Elk1 and/or activate a kinase which phosphorylates Elk1, and consists of an amino acid sequence having at least one amino acid deletion, substitution or addition in an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 and 154.

(4) An isolated polynucleotide comprising a polynucleotide sequence selected from the group consisting of:

(a) a polynucleotide sequence represented by SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151 and 153; and a polynucleotide sequence complementary to said polynucleotide sequence,

(b) a polynucleotide sequence which encodes a protein that acts to phosphorylate Elk1 and/or activate kinase which phosphorylates Elk1, and which hybridizes under stringent conditions with a polynulceotide having a polynulceotide sequence complementary to the polynucleotide sequence of (a); and,

(c) a polynucleotide sequence encoding a protein that acts to phosphorylate Elk1 and/or activate a kinase which phosphorylates Elk1, and consists of a nucleotide sequence having at least one nucleotide deletion, substitution or addition in a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151 and 153.

(5) An isolated polynucleotide comprising a nucleotide sequence which encodes a protein that acts to phosphorylate Elk1 and/or activate a kinase which phosphorylates Elk1, and has at least 95% identity to the polynucleotide sequence according to above item (3) over the entire length thereof.

(6) An isolated polynucleotide comprising a nucleotide sequence which encodes a protein that acts to phosphorylate Elk1 and/or activate a kinase which phosphorylates Elk1, and has at least 95% identity to the polynucleotide sequence according to above item (4) over the entire length thereof.

(7) A purified encoded by the polynucleotide according to any one of above items (3) to (6). The protein is preferably a purified and isolated protein.

(8) A recombinant vector which comprises a polynucleotide according to any one of above items (3) to (6).

(9) A transformed cell which comprises the recombinant vector according to above item (8).

(10) A membrane of the cell according to above item (9), when the protein according to above item (1), (2) or (7) is a membrane protein.

(11) A process for producing a protein comprising:

(a) culturing a transformed cell comprising the isolated polynucleotide according to any one of items (3) to (6) under conditions providing expression of the encoded protein; and

(b) recovering the protein from the culture.

(12) A process for diagnosing a disease or a susceptibility to a disease in a subject related to expression or activity of the protein of item (1), (2) or (7) in a subject comprising:

(a) determining the presence or absence of a mutation in the nucleotide sequence encoding said protein in the genome of said subject; and/or

(b) analyzing the amount of expression of said protein in a sample derived from said subject.

(13) A method for screening a compound for inhibiting or promoting activity for Elk1 phosphorylation action and/or kinase which phosphorylates Elk1, which comprises the steps of:

(a) providing a cell with a gene encoding a protein that acts to phosphorylate Elk1 and/or activate a kinase which phosphorylates Elk1, and a component that provides a detectable signal associated with Elk1 phosphorylation action or kinase which phosphorylates Elk1;

(b) culturing the transformed cell under conditions, which permit the expression of the gene in the transformed cell;

(c) contacting the transformed cell with one or more compounds;

(d) measuring the detectable signal; and

(e) isolating or identifying a compound as an activator compound and/or an inhibitor compound according to the measurement of the detectable signal;

(14) A process for producing a pharmaceutical composition, which comprises the steps of:

(a) providing a cell with a gene encoding a protein that acts to phosphorylate Elk1 and/or activate kinase which phosphorylates Elk1, and a component capable of providing a detectable signal;

(b) culturing a transformed cell under conditions, which permit the expression of the gene in the transformed cell;

(c) contacting the transformed cell with one or more candidate compounds;

(d) measuring the detectable signal;

(e) isolating or identifying a compound as an activator compound and/or an inhibitor compound according to the measurement of the detectable signal; and

(f) optimizing the isolated or identified compound for a pharmaceutical composition.

(15) A kit for screening a compound for inhibiting or promoting activity for Elk1 phosphorylation action and/or a kinase which phosphorylates Elk1, which comprises:

(a) a cell transformed with a gene encoding a protein that acts to phosphorylate Elk1 and/or activate a kinase which phosphorylates Elk1, and a component that provides a detectable signal upon activation of Elk1 phosphorylation action and/or kinase which phosphorylates Elk1; and

(b) reagents for measuring the detectable signal.

(16) A monoclonal or polyclonal antibody that reacts with the protein according to above item (1), (2)or (7).

(17) A process for producing a monoclonal or polyclonal antibody that reacts with the protein of above item (1), (2) or (7), which comprises administering the protein according to above item (1), (2) or (7) as an antigen or epitope-bearing fragments to a non-human animal.

(18) An antisense oligonucleotide complementary to at least a part of the polynucleotide according to any one of above items (3) to (6), which prevents expression of a protein which phosphorylate Elk1 and/or activates kinase which phosphorylates Elk1.

(19) A ribozyme which inhibits activation of Elk1 phosphorylation action and/or a kinase which phosphorylates Elk1 by cleavage of RNA that encodes the protein of above item (1), (2) or (7).

(20) A method for treating a disease, which comprises administering to a subject an effective amount of a compound screened by the process according to above item (13), and/or a monoclonal or polyclonal antibody according to above item (16), and/or an antisense oligonucleotide according to above item (18), and/or a ribozyme according to above item (19) for treating a disease selected from the group consisting of inflammation, autoimmune diseases, cancers, viral infections, GVHD, skin diseases, IgA nephritis, purpuric nephritis, proliferative nephritides, and fulminant hepatitis.

(21) A pharmaceutical composition produced according to item (14) for inhibiting or promoting Elk1 phosphorylation action or a kinase which phosphorylates Elk1.

(22) A pharmaceutical composition according to item (21) for the treatment of inflammation, autoimmune diseases, cancers, viral infections, GVHD, skin diseases, IgA nephritis, purpuric nephritis, proliferative nephritides, and/or fulminant hepatitis.

(23) A method of treating inflammation, autoimmune diseases, cancers, viral infections, GVHD, skin diseases, IgA nephritis, purpuric nephritis, proliferative nephritides, and/or fulminant hepatitis, which comprising administering a pharmaceutical composition produced according to above item (14) to a patient suffering from a disease relating to abnormalities in Elk1 phosphorylation action or kinase which phosphorylates Elk1.

(24) A pharmaceutical composition which comprises a monoclonal or polyclonal antibody according to item (16)as an active ingredient.

(25) A pharmaceutical composition which comprises an antisense oligonucleotide according to item (18) as an active ingredient.

(26) A pharmaceutical composition according to item (24) or (25), wherein a target disease is selected from the group consisting of inflammation, autoimmune diseases, cancers, viral infections, GVHD, skin diseases, IgA nephritis, purpuric nephritis, proliferative nephritides, and fulminant hepatitis.

(27) A computer-readable medium on which a sequence data set has been stored, said sequence data set comprising at least one nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151 and 153 and/or at least one amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 and 154.

(28) A method for calculating identity to other nucleotide sequences and/or amino acid sequences, which comprises comparing data on the medium according to above item (27) with data of said other nucleotide sequences and/or amino acid sequences.

(29) An insoluble substrate to which polynucleotide comprising all or part of the nucleotide sequences selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151 and 153 are fixed.

(30) An insoluble substrate to which polypeptides comprising all or a part of the amino acid sequences selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 and 154 are fixed.

### BRIEF DESCRIPTION OF THE DRAWINGS

FIG. 1 is a graph showing MAP kinase cascade reporter activity inhibition by p38 inhibitor SB203580 in Example 3, the axis of abscissa is SB203580 concentration and the transversal axis is relative luciferase activity.

FIG. 2 indicates the structural formula of staurosporine.

### EXPLANATION OF THE SEQUENCE LISTING

SEQ ID NO: 155 and SEQ ID NO: 156 are primers.

### BEST MODE FOR CARRYING OUT THE INVENTION

At first, in order to further clarify the basic feature of the present invention, the present invention is explained by following how the present invention is completed. In order to obtain a new gene having a function of activating MAP kinase cascade, the following experiments were carried out as shown in the examples. First, using the oligo-capping method, a full-length cDNA was produced from mRNA prepared from normal human lung fibroblasts (purchased from Sanko Junyaku Co., Ltd.), and a full-length cDNA library was constructed in which the cDNA was inserted into the vector pME18S-FL3 (GenBank Accession AB009864). Next, the cDNA library was introduced into E. coli cells, and plasmid preparation was carried out per clone. Then, animal cell expression plasmid pcDNA3.1 (INVITROGEN) comprising DNA encoding human JNK gene, animal cell expression plasmid pcDNA3.1 (INVITROGEN) comprising DNA encoding human p38 gene, plasmid pFA2-Elk1 (STRATAGENE) comprising DNA encoding a fusion protein of part of Elk1 being a substrate of these and GAL4DNA binding region, and reporter plasmid pFR-Luc (STRATAGENE) having a Gal4 recognition site upstream of a gene encoding luciferase were cotransfected with the above full-length cDNA plasmid into HEK293EBNA cells (INVITROGEN). After 24 hours of culture, luciferase activity was measured, and the plasmid with significantly increased luciferase activity compared to that of a control experiment (vector pME18S-FL3 is introduced into a cell in place of a full-length cDNA)was selected (the selected plasmid showed a 5-fold or more increase in luciferase activity compared to that of the control experiment), and the entire nucleotide sequence of the cDNA cloned into the plasmid was determined. As discussed above, since p38, JNK, and ERK etc, existing furthermost downstream of MAP kinase cascade, phosphorylate Elk1 as a good substrate, signal transduction molecules involved in the activation Elk1 phosphorylation action and/or a kinase which phosphorylates Elk1, are highly likely to be signal transduction molecules which activate the MAP kinase cascade. Therefore, the protein encoded by the cDNA thus obtained is a signal molecule involved in the phosphorylation of Elk1 and/or a kinase which phosphorylates Elk1, and at the same time, can be expected to be a signal transduction molecule involved in the activation of the MAP kinase cascade.

The phrase “activate kinase which phosphorylates Elk1” includes both direct modification and activation of a kinase which phosphorylates Elk1, and indirect activation of the kinase by activating upstream signal(s).

The present invention is described in detail below.

In the present invention, the words “acts to phosphorylate Elk1” or “acts to activate a kinase which phosphorylates Elk1” refers to having an action which phosphorylates Elk1 directly or indirectly when a gene is introduced into a suitable cell and the protein encoded by the gene is excessively expressed. Phosphorylation of Elk1 can be measured, for example, by a reporter gene assay comprising cotransfecting into a cell together with a gene resulting from fusion of Elk1 and GAL4DNA binding region, a reporter gene wherein a luciferase gene is linked downstream of a GAL4 recognition sequence. In the assay, phosphorylation can be evaluated by an increase in reporter activity in cells into which the gene was introduced, compared to control cells (cells into which a null vector only was introduced). Increase in reporter activity is preferably by a factor of 1.5 or more, more preferably by a factor of 2 or more, and still more preferably by a factor of 5 or more.

Reporter activity can be measured by cloning a polynucleotide (e.g. cDNA) encoding the protein to be expressed into a suitable expression vector, co-transfecting the thus prepared expression vector into a suitable cell together with a gene resulting from fusion of Elk1 and GAL4DNA binding region, and a reporter gene wherein a luciferase gene is linked downstream of a GAL4 recognition sequence, and after culturing for a certain period, then measuring the reporter activity. Suitable expression vectors are well known to those skilled in the art, and examples include pME18S-FL3, pcDNA3.1 (Invitrogen). The reporter gene can be one which enables a person skilled in the art to easily detect the expression thereof, and examples include a gene encoding luciferase, chloramphenicol acetyl transferase, or β-galactosidase. Use of a gene encoding luciferase is most preferable, and examples of a gene resulting from fusion of Elk1 and GAL4DNA binding region include pFA2-Elk1 (STRATAGENE), and examples of a reporter gene wherein a luciferase gene is linked downstream of a GAL4 recognition sequence include pFR-Luc (STRATAGENE). Suitable cells include cells which exhibit an MAPK cascade activation response to stimulation by IL-1, TNF-α or the like. Examples include 293-EBNA cells. Cell culture and introduction of genes into cells (transfection) can be performed and optimized by a person skilled in the art by known techniques.

As a preferable method, 293-EBNA cells are inoculated on 5% FBS (Fetal Bovine Serum)-containing DMEM (Dulbecco's Modified Eagle Medium) medium in a 96-well cell culture plate to a final cell density of 1×10⁴ cells/well, and cultured for 24 hours at 37° C., in the presence of 5% CO₂. Then, pFR-Luc (STRATAGENE), pFA2-Elk1 (STRATAGENE), pcDNA3.1 (+) (INVITROGEN) into which human p38 gene has been incorporated, pcDNA3.1 (+) (INVITROGEN) into which human JNJ1 β 1 gene has been incorporated, and the expression vector are co-transfected into the cells in a well using FuGENE 6 (Roche). After 24 hours of culture at 37° C., phosphorylation of Elk1 or activation of a kinase that phosphorylates Elk1 is then measured by measuring luciferase activity using a long term luciferase assay system, Picagene LT2.0 (Toyo Ink). For example, luciferase activity can be measured using PerkinElmer's Wallac ARVOTMST 1420 MULTILABEL COUNTER. The method for gene introduction by FuGENE6, and measurement of luciferase activity by Picagene LT2.0 can be performed respectively according to the attached protocols. In a method of gene introduction with a 96-well plate using FuGENE6, the amount of FuGENE6 per 1 well is suitably 0.3 to 0.5 μl, preferably 0.5 μl. The respective amounts per 1 well of the plasmid genes to be introduced are: pFR-Luc plasmid amount, 50 to 100 ng, preferably 60 ng; pFA2-Elk1 plasmid amount, 0.1 to 0.5 ng, preferably 0.25 ng; amount of pcDNA3.1 (+) plasmid into which human p38 gene has been incorporated, 5 ng; and amount of pcDNA3.1(+) plasmid into which human JNJ1 β 1 gene has been incorporated, 30 ng. An ability to phosphorylate Elk1 or to activate a kinase that phosphorylates Elk1 refers to an ability to increase the reporter activity (luciferase activity) relative to the control experiment (using cells into which a null vector only was introduced). Increase in reporter activity is preferably by a factor of 1.5 or more, more preferably by a factor of 2 or more, and still more preferably by a factor of 5 or more.

Related to the amino acid sequences of SEQ ID NOS. 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 and 154, the present invention provides for a protein that:

(a) comprises any one of the above amino acid sequence;

(b) is a polypeptide having one of the above amino acid sequences;

(c) phosphorylates Elk1 and/or activates kinase which phosphorylates Elk1 and consists of an amino acid sequence having at least one amino acid, preferably several amino acids deletion, substitution or addition in any one of the above amino acid sequences; and

(d) comprises an amino acid sequence, which has at least 95% identity, preferably at least 97-99% identity, to any one of the amino acid sequences of SEQ ID NOS. 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 and 154, over the entire length thereof.

“Identity” as known in the art, is a relationship between two or more protein sequence or two or more polynucleotide sequences, as determined by comparing the sequences. In the art, “identity” also means the degree of sequence relatedness between protein or polynucleotide sequences, as determined by the match between protein or polynucleotide sequences, as the case may be, as determined by the match between strings of such sequences. “Identity” and “similarity” can be readily calculated by known methods. Preferred methods to determine identity are designed to give the largest match between the sequences tested. Methods to determine identity and similarity are codified in publicly available computer programs. “Identity” can be determined by using, for example, the BLAST (Basic Local Alignment Search Tool) program (for example, Altschul S F, Gish W, Miller W, Myers E W, Lipman D J., J. Mol. Biol., 215: p403-410(1990), Altschul S F, Madden T L, Schaffer A A, Zhang Z, Miller W, Lipman D J,. Nucleic Acids Res. 25: p3389-3402 81997), however methods of determining identity are not limited to this. Where software such as BLAST is used, it is preferable to use default values.

The main initial conditions generally used in a BLAST search are as follows, but are not limited to these. An amino acid substitution matrix is a matrix numerically representing the degree of analogy of each pairing of each of the 20 types of amino acid, and normally the default matrix, BLOSUM62, is used. The theory of this amino acids substitution matrix is shown in Altschul S. F., J. Mol. Biol. 219: 555-565 (1991), and its applicability to DNA sequence comparison is shown in States D. J., Gish W., Altschul S. F., Methods, 3: 66-70 (1991). In this case, optimal gap cost is determined empirically and in the case of BLOSUM62, preferably parameters, Existence 11, Extension 1 are used.

The expected value (EXPECT) is the threshold value concerning statistical significance for a match with a database sequence, and the default value is 10.

The Examples described below demonstrate that the protein consisting of any one of the amino acid sequences of the above SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 and 154, is capable of phosphorylating Elk1 action and/or activating kinase which phosphorylates Elk1.

Related to the polynucleotide sequences of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151 and 153, the present invention further provides an isolated polynucleotide that:

(a) comprises a nucleotide sequence, which has at least 95% identity, preferably at least 97-99% identity to any one of the above sequences;

(b) has a polynucleotide sequence encoding a protein which has at least 97-99% identity, to any one of the amino acid sequences of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 and 154.

Polynucleotides which are identical or almost identical to a nucleotide sequences contained in the above nucleotide sequence may be used as hybridization probes or as primers for a nucleic acid amplification reaction to isolate full-length cDNAs and genomic clones encoding proteins of the present invention or cDNA and genomic clones of other genes that have a high sequence similarity to the above sequences. Typically, these nucleotide sequences are 70% identical, preferably 80% identical, more preferably 90% identical, most preferably 95% identical to the above sequences. The probes or primers will generally comprises at least 15 nucleotides, preferably 30 nucleotides and may have 50 nucleotides. Particularly preferred probes will have between 30 and 50 nucleotides. Particularly preferred primers have between 20 and 25 nucleotides.

The polynucleotide of the present invention may be either in the form of a DNA such as cDNA, a genomic DNA obtained by cloning or synthetically produced, or may be in the form of RNA such as mRNA. The polynucleotide may be single-stranded or double-stranded. The double-stranded polynucleotides may be double-stranded DNA, double-stranded RNA or DNA:RNA hybrid. The single-stranded polynucleotide may be sense strand also known as coding strand or antisense strand also known as non-coding strand.

Those skilled in the art can prepare a protein having the same Elk1 phosphorylating activity and/or kinase activity which phosphorylates Elk1 as the protein having an amino acid sequence of any one of SEQ ID NOS 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 and 154, by means of appropriate substitution of an amino acid in the protein using known methods. One such method involves conventional mutagenesis procedures using mutagens for the DNA encoding the protein. Another method is, for example, site-directed mutagenesis (e.g., Mutan-Super Express Km Kit from Takara Shuzo Co., Ltd.). Mutations of amino acids in proteins may also occur in nature. Thus, the present invention also includes a mutated protein which is capable of phosphorylating Elk1 and/or activating kinase which phosphorylates Elk1 and which has at least one amino acid deletion, substitution or addition compared to the corresponding amino acid sequence of SEQ ID NO 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 or 154. The number of mutations is preferably up to 10, more preferably up to 5, most preferably up to 3.

Examples of such amino acid substitutions include substitutions within the following groups: (glycine, alanine), (valine, isoleucine, leucine), (aspartic acid, glutamic acid), (asparagine, glutamine), (serine, threonine), (lysine, arginine) and (phenylalanine, tyrosine).

Based on a nucleotide sequence (e.g., SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151 or 153) encoding a protein consisting of an amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 or 154, or fragments thereof, those skilled in the art can routinely isolate a DNA with a high sequence similarity to any one of these nucleotide sequences by using hybridization techniques and the like, and obtain proteins having the same Elk1 phosphorylating activity and/or kinase activity which phosphorylates Elk1 as the protein having an amino acid sequence of SEQ ID NOS 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 or 154. Thus, the present invention also includes a protein that phosphorylates Elk1 and/or activates kinase which phosphorylates Elk1 and comprises an amino acid sequence having a high identity to the amino acid sequence of above SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 or 154. “High identity” refers to an amino acid sequence having an identity of at least 90%, preferably at least 97-99% over the entire length of any one of the above SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 and 154.

The proteins of the present invention may be natural proteins derived from any human or animal cells or tissues, chemically synthesized proteins, or proteins obtained by genetic recombination techniques. The protein may or may not be subjected to post-translational modifications such as sugar chain addition or phosphorylation.

The present invention also includes a polynucleotide encoding the above protein of the present invention. Examples of nucleotide sequences encoding a protein consisting of an amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 or 154 include a nucleotide sequences of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151 or 153. The DNA includes cDNA, genomic DNA, and chemically synthesized DNA. In accordance with the degeneracy of the genetic code, at least one nucleotide in the nucleotide sequence encoding a protein consisting of an amino acid sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 or 154 can be substituted with other nucleotides without altering the amino acid sequence of the protein produced from the gene. Therefore the DNA sequences of the present invention also include nucleotide sequences altered by substitution based on the degeneracy of the genetic code without alteration of the amino acid sequence. Such DNA sequences can be synthesized using known methods.

The DNA of the present invention includes a DNA which encodes a protein capable of phosphorylating Elk1 and/or activating kinase which phosphorylates Elk1 and hybridizes under stringent conditions with the DNA sequence of any one of the above nucleotide sequences of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151 and 153 or a polynucleotide sequence complementary to said nucleotide sequence. Stringent conditions are apparent to those skilled in the art, and can be easily attained in accordance with various laboratory manuals such as T. Maniatis et al., Molecular Cloning A Laboratory Manual, Cold Spring Harbor Laboratory 1982, 1989.

That is, “stringent conditions” refer to overnight incubation at 37° C. in a hybridization solution containing 30% formamide, 5×SSC (0.75 M NaCl, 75 mM trisodium citrate),5×Denhardt's solution, 0.5% SDS, 100 μg/ml denatured, sheared salmon sperm DNA) followed by washing (three times) in 2×SSC, 0.1% SDS for 10 minutes at room temperature, then followed by washing (two times) in 1×SSC, 0.1% SDS for 10 minutes at 37° C.(low stringency). Preferred stringent conditions are overnight incubation at 42° C. in a hybridization solution containing 40% formamide, followed by washing (three times) in 2×SSC, 0.1% SDS for 10 minutes at room temperature, then followed by washing (two times) in 0.2×SSC, 1% SDS for 10 minutes at 42° C.(moderate stringency). More preferred stringent conditions are overnight incubation at 42° C. in a hybridization solution containing 50% formamide, followed by washing (three times) in 2×SSC, 0.1% SDS for 10 minutes at room temperature, followed by washing (two times) in 0.2×SSC, 0.1% SDS for 10 minutes at 50° C. (high stringency). The DNA sequence thus obtained must encode a protein capable of phosphorylating Elk1 and/or activating kinase which phosphorylates Elk1.

The present invention also includes a polynucleotide comprising a nucleotide sequence which encodes a protein capable of phosphorylating Elk1 and/or activating kinase which phosphorylates Elk1 and has a high sequence similarity to the nucleotide sequence of the polynucleotide according to above item (3) or (4). Typically these nucleotide sequence are 95% identical, preferably 97% identical, more preferably 98-99% identical, most preferably at least 99% identical to the nucleotide sequence of the polynucleotide according to above item (3) or (4) over the entire length thereof.

The above nucleotide sequence of the present invention can be used to produce the above protein using recombinant DNA techniques. In general, the DNA and peptide of the present invention can be obtained by:

(A) cloning the DNA encoding the protein of the present invention;

(B) inserting the DNA encoding the entire coding region of the protein or a part thereof into an expression vector to construct a recombinant vector;

(C) transforming host cells with the recombinant vector thus constructed; and

(D) culturing the obtained cells to express the protein or its analogue, and then purifying it by column chromatography.

General procedures necessary to handle DNA and recombinant host cells (e.g., E. coli) in the above steps are well known to those skilled in the art, and can be easily carried out in accordance with various laboratory manuals such as T. Maniatis et al., supra. All the enzymes, reagents, etc., used in these procedures are commercially available, and unless otherwise stated, such commercially available products can be used according to the conditions specified by the manufactures' instructions to attain completely its objects. The above steps (A) to (D) can be further illustrated in more details as follows.

Techniques for cloning the DNA encoding the protein of the above step (A) include, in addition to the methods described in the specification of the present application, PCR amplification using synthetic DNA containing a part of the nucleotide sequence of the present invention (e.g., SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151 or 153) as a primer, and selection of the DNA inserted into a suitable vector by hybridization with a labeled DNA fragment encoding a partial or full coding region of the protein of the present invention or a labeled synthetic DNA. Another technique involves direct amplification of DNA from total RNAs or mRNA fractions prepared from cells or tissues, using the reverse transcriptase polymerase chain reaction (RT-PCR method). As a DNA inserted into a suitable vector, for example, a commercially available library (e.g., from CLONTECH and STRATAGENE) can be used.

Techniques for hybridization are normally used in the art, and can be easily carried out in accordance with various laboratory manuals such as T. Maniatis et al., supra.

Depending on the intended purpose, the cloned DNA encoding the protein of the present invention can be used as such or if desired after digestion with a restriction enzyme or addition of a linker. The DNA thus obtained may have a nucleotide sequence of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151 or 153, or a polynucleotide of above items (3) to (6). The DNA sequence to be inserted into an expression vector in the above step (B) may be a full-length cDNA or a DNA fragment encoding the above full-length protein, or a DNA fragment constructed so that it expresses a part thereof.

Thus, the present invention also includes a recombinant vector, which comprises the above DNA sequence. The expression vector for the protein of the present invention can be produced, for example, by excising the desired DNA fragment from the DNA encoding the protein of the present invention, and ligating the DNA fragment downstream of a promoter in a suitable expression vector.

Expression vectors for use in the present invention may be any vectors derived from prokaryotes (e.g., E. coli), yeast, fungi, insect viruses and vertebrate viruses so long as such vectors are replicable. However, the vectors should be selected to be compatible with microorganisms or cells used as hosts. Suitable combinations of host cell—expression vector systems are selected depending on the desired expression product.

When bacteria are used as hosts, plasmid vectors compatible with these bacteria are generally used as replicable expression vectors for recombinant DNA molecules.

For example, the plasmids pBR322 and pBR327 can be used to transform E. coli. Plasmid vectors normally contain an origin of replication, a promoter, and a marker gene conferring upon a recombinant DNA a phenotype useful for selecting the cells transformed with the recombinant DNA. Example of such promoters include a β-lactamase promoter, lactose promoter and tryptophan promoter. Examples of such marker genes include an ampicillin resistance gene, and a tetracycline resistance gene. Examples of suitable expression vectors include the plasmids pUC18 and pUC19 in addition to pBR322, pBR327.

In order to express the DNA of the present invention in yeast, for example, YEp24 can be used as a replicable vector. The plasmid YEp24 contains the URA3 gene, which can be employed as a marker gene. Examples of promoters in expression vectors for yeast cells include promoters derived from genes for 3-phosphoglycerate kinase, glyceraldehyde-3-phosphate dehydrogenase and alcohol dehydrogenase.

Examples of promoters and terminators for use in expression vectors to express the DNA of the present invention in fungal cells include promoters and terminators derived from genes for phosphoglycerate kinase (PGK), glyceraldehyde-3-phosphate dehydrogenase (GAPD) and actin. Examples of suitable expression vectors include the plasmids pPGACY2 and pBSFAHY83.

Examples of promoters for use in expression vectors to express the DNA of the present invention in insect cells include a polyhedrin promoter and P10 promoter.

Recombinant vectors used to express the DNA of the present invention in animal cells normally contain functional sequences to regulate genes, such as an origin of replication, a promoter to be placed upstream of the DNA of the present invention, a ribosome-binding site, a polyadenylation site and a transcription termination sequence. Such functional sequences, which can be used to express the DNA of the present invention in eukaryotic cells, can be obtained from viruses. Examples of such functional sequences include an SR α promoter, SV40 promoter, LTR promoter, CMV (cytomegalovirus) promoter and HSV-TK promoter. Among them, a CMV promoter and SR α promoter can be preferably used. Promoters to be placed inherently upstream of the gene encoding the protein of the present invention, can be used so long as they are suitable for use in the above host-vector systems. Examples of origins of replication include foreign origins of replication, for example, those derived from viruses such as adenovirus, polyoma virus and SV40 virus. When vectors capable of integration into host chromosomes are used as expression vectors, origins of replication of the host chromosomes may be employed. Examples of suitable expression vectors include the plasmids pSV-dhfr (ATCC 37146), pBPV-1(9-1) (ATCC 37111), pcDNA3.1 (INVITROGEN) and pME18S-FL3.

The present invention also includes a transformed cell, which comprises the above recombinant vector.

Microorganisms or cells transformed with the replicable recombinant vector of the present invention can be selected from remaining untransformed parent cells based on at least one phenotype conferred by the recombinant vector. Phenotypes can be conferred by inserting at least one marker gene into the recombinant vector. Marker genes naturally contained in replicable vectors can be employed. Examples of marker genes include drug resistance genes such as neomycin resistance genes, and genes encoding dihydrofolate reductase.

As hosts for use in the above step (C), any of prokaryotes (e.g., E. coli), microorganisms (e.g., yeast and fungi) as well as insect and animal cells can be used so long as such hosts are compatible with the expression vectors used. Examples of such microorganisms include Escherichia coli strains such as E. coli K12 strain 294 (ATCC 31446), E. coli X1776 (ATCC 31537), E. coli C600, E. coli JM109 and E. coli B strain; bacterial strains belonging to the genus Bacillus such as Bacillus subtilis; intestinal bacteria other than E. coli, such as Salmonella typhimurium or Serratia marcescens; and various strains belonging to the genus Pseudomonas. Examples of such yeast include Saccharomyces cerevisiae, Schizosaccharomyces pombe, and Pichia pastoris. Examples of such fungi include Aspergillus nidulans, and Acremonium chrysogenum (ATCC 11550).

As insect cells, for example, Spodoptera frugiperda (Sf cells), High Five™ cells derived from eggs of Trichoplusiani, etc., can be used when the virus is AcNPV. Examples of such animal cells include HEK 293 cells, COS-1 cells, COS-7 cells, Hela cells, and Chinese hamster ovary (CHO) cells. Among them, CHO cells and HEK 293 cells are preferred.

When cells are used as hosts, combinations of expression vectors and host cells to be used vary with experimental objects. According to such combinations, two types of expression (i.e. transient expression and constitutive expression) can be included.

“Transformation” of microorganisms and cells in the above step (C) refers to introducing DNA into microorganisms or cells by forcible methods or phagocytosis of cells and then transiently or constitutively expressing the trait of the DNA in a plasmid or an intra-chromosome integrated form. Those skilled in the art can carry out transformation by known methods [see e.g., “Idenshi Kougaku Handbook (Genetic Engineering Handbook)”, an extra issue of “Jikken Igaku (Experimental Medicine)”, YODOSHA CO., LTD.]. For example, in the case of animal cells, DNA can be introduced into cells by known methods such as DEAE-dextran method, calcium-phosphate-mediated transfection, electroporation, lipofection, etc. For stable expression of the protein of the present invention using animal cells, there is a method in which selection can be carried out by clonal selection of the animal cells containing the chromosomes into which the introduced expression vectors have been integrated. For example, transformants can be selected using the above selectable marker as an indication of successful transformation. In addition, the animal cells thus obtained using the selectable marker can be subjected to repeated clonal selection to obtain stable animal cell strains highly capable of expressing the protein of the present invention. When a dihydrofolate reductase (DHFR) gene is used as a selectable marker, one can culture animal cells while gradually increasing the concentration of methotrexate (MTX) and select the resistant strains, thereby amplifying the DNA encoding the protein of the present invention together with the DHFR gene to obtain animal cell strains having higher levels of expression.

The above transformed cells can be cultured under conditions which permit the expression of the DNA encoding the protein of the present invention to produce and accumulate the protein of the present invention. In this manner, the protein of the present invention can be produced. Thus, the present invention also includes a process for producing a protein, which comprises culturing a transformed cell comprising the isolated polynucleotide according to above item (3) to (6) under conditions providing expression of the encoded protein and recovering the protein from the culture.

The above transformed cells can be cultured by methods known to those skilled in the art (see e.g., “Bio Manual Series 4”, YODOSHA CO., LTD.). For example, animal cells can be cultured by various known animal cell culture methods including attachment culture such as Petri dish culture, multitray type culture and module culture, attachment culture in which cells are attached to cell culture carriers (microcarriers), suspension culture in which productive cells themselves are suspended. Examples of media for use in the culture include media commonly used for animal cell culture, such as D-MEM and RPMI 1640.

In order to separate and purify the protein of the present invention from the above culture, suitable combinations of per se known separation and purification methods can be used. Examples such methods include methods based on solubility, such as salting-out and solvent precipitation; methods based on the difference in charges, such as ion-exchange chromatography; methods mainly based on the difference in molecular weights, such as dialysis, ultrafiltration, gel filtration and SDS-polyacrylamide gel electrophoresis; methods based on specific affinity, such as affinity chromatography; methods based on the difference in hydrophobicity, such as reverse phase high performance liquid chromatography; and methods based on the difference in isoelectric points, such as isoelectric focusing. For example, a protein of the present invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxyapatite chromatography and lectin chromatography. Well known techniques for refolding proteins may be employed to regenerate active conformation when the polypeptide is denatured during intracellular synthesis, isolation or purification.

The protein of the present invention can also be produced as a fusion protein with another protein. These fusion proteins are also included within the present invention. For the expression of such fusion proteins, any vectors can be used so long as the DNA encoding the protein can be inserted into the vectors and the vectors can express the fusion protein. Examples of proteins to which a polypeptide of the present invention can be fused include glutathione S-transferase (GST) and a hexa-histidine sequence (6×His). The fusion protein of the protein of the present invention with another protein can be advantageously purified by affinity chromatography using a substance with an affinity for the fusion partner protein. For example, fusion proteins with GST can be purified by affinity chromatography using glutathione as a ligand.

The present invention also includes an inhibitory protein, i.e., a protein capable of inhibiting the activity of the protein of above item (7). Examples of such inhibitory proteins include antibodies, thereby inhibiting the expression of their activity.

The present invention also includes an antibody that reacts with the protein of the present invention or a fragment thereof. More preferably, the present invention relates to an antibody that reacts specifically with the above-mentioned protein of the present invention or a fragment thereof, and the method of production of such an antibody. Herein, “specifically” refers to there being little, or preferably no, crossreactivity. The antibody is not specifically limited so long as it can recognize the protein of the present invention. Examples of such antibodies include polyclonal antibodies, monoclonal antibodies and their fragments, single chain antibodies and humanized antibodies. Antibody fragments can be produced by known techniques. Examples of such antibody fragments include, but not limited to, F(ab′)₂ fragments, Fab′ fragments, Fab fragments and Fv fragments. The antibody that specifically binds the protein of the present invention can be produced using the protein of the present invention or a peptide thereof as an immunogen according to per se known process for producing antibodies or antisera. For example, a monoclonal or polyclonal antibody can be produced by administering the protein or epitope-bearing fragments according to above item (1) or (2) as an antigen to a non-human animal. Such methods are described, for example, in “Shin Idenshi Kougaku Handbook (New Genetic Engineering Handbook)”, the third edition, an extra issue of “Jikken Igaku (Experimental Medicine)”, YODOSHA CO., LTD.

In the case of polyclonal antibodies, for example, the protein of the present invention or a peptide thereof can be injected to animals to produce antibodies directed against the protein or peptide, and then their blood can be collected. The polyclonal antibodies can be purified from the blood, for example, by ammonium sulfate precipitation or ion-exchange chromatography, or by using the affinity column on which the antigen protein has been immobilized.

In the case of monoclonal antibodies, for example, animals such as mice are immunized with the protein of the present invention, their spleen is removed and homogenized to obtain spleen cells, which are then fused with mouse myeloma cells by using a reagent such as polyethylene glycol. From the resulting hybrid cells (i.e. hybridoma cells), the clone producing the antibody directed against the protein of the present invention can be selected. Then, the resulting clonal hybridoma cells can be implanted intraperitoneally into mice, the ascitic fluid recovered from the mice. The resulting monoclonal antibody can be purified from ascitic fluid, for example, by ammonium sulfate precipitation or ion-exchange chromatography, or by using the affinity column on which the protein has been immobilized.

When the resulting antibody is used to administer it to humans, it is preferably used as a humanized antibody or human antibody in order to reduce its immunogenicity. The human antibody can be produced using transgenic mice or other mammals. For a general review of humanized, and human antibodies, see, for example, Morrison, S. L. et al., Proc. Natl. Acad. Sci. USA, 81:6851-6855 (1984); Jones, P. T. et al., Nature 321:522-525 (1986); Hiroshi Noguchi, Igaku no Ayumi (J. Clin. Exp. Med.) 167:457-462 (1993); Takashi Matsumoto, Kagaku to Seibutsu (Chemistry and Biology) 36:448-456 (1998). Humanized chimeric antibodies can be produced by linking a V region of a mouse antibody to a C region of a human antibody. Humanized antibodies can be produced from a mouse monoclonal antibody by substituting a sequence derived from a human antibody for a region other than a complementarity-determining region. In addition, human antibodies can be directly produced in the same manner as the production of conventional monoclonal antibodies by immunizing the mice whose immune systems have been replaced with human immune systems. These antibodies can be used to isolate or to identify clones expressing the protein or to purify the protein of the present invention from a cell extract or transformed cells producing the protein of the present invention. These antibodies can also be used to construct ELISA, RIA (radioimmunoassay) and western blotting systems. These assay systems can be used for diagnostic purposes for detecting an amount of the protein of the present invention present in a body sample in a tissue or a fluid in the blood of an animal, preferably human. For example, they can be used for diagnosis of a disease characterized by undesirable activation of MAP kinase cascade resulting from (expression) abnormality of the protein of the present invention, such as inflammation, autoimmune disease, infection (for example, HIV infection), cancer and the like. In order to provide a basis for diagnosis of a disease, a standard value must be established. This is a well-known technique to those skilled in the art. For example, a method of calculating the standard value comprises binding a body fluid or a cell extract of normal individual of a human or an animal to an antibody against the protein of the present invention under a suitable condition for the complex formation, detecting the amount of the antibody-protein complex by chemical or physical means and then calculating the standard value for the normal sample using a standard curve prepared from a standard solution containing a known amount of an antigen (the protein of the present invention). The presence of a disease can be confirmed by deviation from the standard value obtained by comparison of the standard value with the value obtained from a sample of an individual latently suffering from a disease associated with the protein of the present invention. These antibodies can also be used as reagents for studying functions of the protein of the present invention.

The antibodies of the present invention can be purified and then administered to patients characterized by undesirable activation of MAP kinase cascade resulting from (expression) abnormality of the protein of the present invention, such as inflammation, autoimmune disease, infection (such as HIV infection), cancer and the like. Thus in another aspect, the present invention is a pharmaceutical composition which comprises the above antibody as an active ingredient, and therapy using the antibody of the present invention. In such pharmaceutical compositions, the active ingredient may be combined with other therapeutically active ingredients or inactive ingredients (e.g., conventional pharmaceutically acceptable carriers or diluents such as immunogenic adjuvants) and physiologically non-toxic stabilizers and excipients. The resulting combinations can be sterilized by filtration, and formulated into vials after lyophilization or into various dosage forms in stabilized and preservable aqueous preparations. Administration to a patient can be intra-arterial administration, intravenous administration and subcutaneous administration, which are well known to those skilled in the art. The dosage range depends upon the weight and age of the patient, route of administration and the like. Suitable dosages can be determined by those skilled in the art. These antibodies exhibit therapeutic activity by inhibiting the MAP kinase activation mediated by the protein of the present invention.

The DNA of the present invention can also be used to isolate, identify and clone other proteins involved in intracellular signal transduction processes. For example, the DNA sequence encoding the protein of the present invention can be used as a “bait” in yeast two-hybrid systems (see e.g., Nature 340:245-246 (1989)) to isolate and clone the sequence encoding a protein (“prey”) which can associate with the protein of the present invention. In a similar manner, it can be determined whether the protein of the present invention can associate with other cellular proteins (e.g., JNK, p38). In another method, proteins which can associate with the protein of the present invention can be isolated from cell extracts by immunoprecipitation [see e.g., “Shin Idenshi Kougaku Handbook (New Genetic Engineering Handbook)”, an extra issue of “Jikken Igaku (Experimental Medicine)”, YODOSHA CO., LTD.] using antibodies directed against the protein of the present invention. In still another method, the protein of the present invention can be expressed as a fusion protein with another protein as described above, and immunoprecipitated with an antibody directed against the fusion protein in order to isolate a protein which can associate with the protein of the present invention.

The above-described diagnostic assays offer a process for diagnosing or determining a susceptibility to the diseases through detection of mutation in the gene encoding protein (1) or (2) which functions to phosphorylate Elk1 and/or activate a kinase which phosphorylates Elk1, by the methods described. In addition, such diseases may be diagnosed by methods comprising determining from a sample derived from a subject an abnormally decreased or increased level of protein or mRNA. Decreased or increased expression can be measured at the mRNA level using any of the methods well known in the art for the quantitation of polynucleotides, such as nucleic amplification, for example, RT-PCR, RNase protection, Northern blotting and other hybridization methods. Assay techniques that can be used to determine levels of a protein in a sample derived from a host are well-known to those skilled in the art. Such assay methods include radioimmunoassays, competitive-binding assays, Western blot analysis and ELISA assays. The DNA of the present invention can be used to detect abnormality in the DNA or mRNA encoding the protein of the present invention or a peptide fragment thereof. Thus, for example, the DNA of the present invention is useful for gene diagnosis regarding damage, mutations, and reduced, increased or over-expression of the DNA or mRNA. That is, the present invention includes a method for diagnosing a disease or susceptibility to a disease related to expression or activity of the protein in a subject, which comprises:

(a) determining the presence or absence of a mutation in the nucleotide sequence encoding the protein of claim 1 or 2 in the genome of said subject; and/or

(b) analyzing the amount of expression of said protein in a sample derived from said subject, wherein a diagnosis of disease preferably is made when the amount of the protein expressed is 2-fold or higher than normal, or half or lower than normal.

When the nucleotide sequence encoding protein (1) or (2) which functions to phosphorylate Elk1 and/or activate a kinase which phosphorylates Elk1, contains a mutation according to the above step (a), the mutation may cause disease associated with the MAP kinase cascade. When the amount of the expression of the protein of above item (12) is different from the normal value according to the above step (b), abnormal expression of the novel protein of the present invention which functions to phosphorylate Elk1 and/or activate a kinase which phosphorylates Elk1 may be responsible for diseases associated with the Elk1 phosphorylating action or a kinase which phosphorylates Elk1. Determination of the presence or absence of a mutation in the nucleotide sequence encoding protein (1) or (2) which functions to phosphorylate Elk1 and/or activate a kinase which phosphorylates Elk1 in the above step (a) may involve RT-PCR using a part of the nucleotide sequence of protein (1) or (2) encoding gene as a primer, followed by conventional DNA sequencing to detect the presence or absence of the mutation. PCR-SSCP [Genomics 5:874-879 (1989); “Shin Idenshi Kougaku Handbook (New Genetic Engineering Handbook)”, an extra issue of “Jikken Igaku (Experimental Medicine)”, YODOSHA CO., LTD.] can also be used to determine the presence or absence of the mutation. Measurement of the amount of the expression of the protein in the above step (b) may involve, for example, using the antibody of above item (16).

The present invention also includes a method for screening compounds for activity as inhibitors or activators of MAP kinase cascade, which comprises the steps of:

(a) providing a cell with a gene encoding a protein that phosphorylates Elk1 and/or activates kinase which phosphorylates Elk1, and a component that provides a detectable signal upon activation thereof;

(b) culturing the transformed cell under conditions, which permit the expression of the gene in the transformed cell;

(c) contacting the transformed cell with one or more compounds; and

(d) measuring the detectable signal; and

(e) isolating or identifying a compound as an activator compound and/or an inhibitor compound according to the measurement of the detectable signal.

Preferably, in step (e), a compound that increases said detectable signal 2-fold or higher than normal is isolated or identified as an activator compound, and a compound that decreases said detectable signal to half or lower than normal is isolated or identified as an inhibitor compound.

Examples of components capable of providing a detectable signal include reporter genes. Reporter genes are used instead of directly detecting the activation of transcription factors of interest. The transcriptional activity of a promoter of a gene is analyzed by linking the promoter to a reporter gene and measuring the activity of the product of the reporter gene (“Bio Manual Series 4” (1994), YODOSHA CO., LTD.). Any peptide or protein can be used so long as those skilled in the art can measure the activity or amount of the expression product (including the amount of the produced mRNA) of the reporter genes. For example, enzymatic activity of chloramphenicol acetyltransferase, β-galactosidase, luciferase, etc., can be measured. Any reporter plasmids can be used to evaluate MAP kinase cascade activation. One example is the reporter plasmids that have a yeast transcription factor Gal4 recognition sequence inserted upstream of the reporter gene. For example, pFR-Luc (STRATAGENE) can be used by introduction into a host cell together with various fusion proteins of genes being substrates of MAP kinase such as Elk1 and ATF2 connected to yeast Gal4 protein DNA binding region gene: e.g. pFA2-Elk1 or pFA2-ATF2 (STRATAGENE).

Any host cells can be used so long as Elk1 phosphorylation action or kinase activation which phosphorylates Elk1 can be detected in the host cells. Preferred host cells are mammalian cells such as HEK293-EBNA cells. Transformation and culture of the cells can be carried out as described above. In a specific embodiment, the method for screening a compound which inhibits or activates MAP kinase cascade activation comprises culturing the transformed cell for a certain period of time, adding a certain amount of a test compound, measuring the reporter activity expressed by the cell after a certain period of time, and comparing the activity with that of a cell to which the test compound has not been added. The reporter activity can be measured by methods known in the art (see e.g., “Bio Manual Series 4” (1994), YODOSHA CO., LTD.). Examples of test compounds include, but not limited to, low molecular weight compounds and peptides. Test compounds may be artificially synthesized compounds or naturally occurring compounds. Test compounds may be a single compound or mixtures. Other examples of methods for detecting detectable signals include ELISA assay or Western blotting assay using an antibody to detect phosphorylation of various MAP kinases such as p38, JNK, ERK, etc., in addition to assays of the above reporter genes.

It is also possible to produce a pharmaceutical composition according to the following steps (a) to (f):

(a) providing a cell with a gene encoding a protein that phosphorylates Elk1 and/or activates kinase which phosphorylates Elk1, and a component that provides a detectable signal upon activation thereof;

(b) culturing the transformed cell under conditions, which permit the expression of the gene in the transformed cell;

(c) contacting the transformed cell with one or more candidate compounds;

(d) measuring the detectable signal; and

(e) isolating or identifying a compound as an activator compound and/or an inhibitor compound according to the measurement of the detectable signal; and

(f) optimizing the isolated or identified compound as a pharmaceutical composition.

Preferably, in step (e), a compound that increases said detectable signal 2-fold or higher than normal is isolated or identified as an activator compound, and a compound that decreases said detectable signal to half or lower than normal is isolated or identified as an inhibitor compound.

The amount of mRNA can be measured, for example, by northern hybridization, RT-PCR, etc. The amount of proteins can be measured, for example, by using antibodies. The antibodies may be produced by known methods. Commercially available antibodies(from, e.g., Wako Pure Chemical Industries, Ltd.)can also be used.

The protein of the present invention may also be used in a method for the structure-based design of an agonist, antagonist or inhibitor of the protein, by:

(a) determining the three-dimensional structure of the protein;

(b) deducing the three-dimensional structure for the likely reactive or binding site(s) of an agonist, antagonist or inhibitor;

(c) synthesizing candidate compounds that are predicted to bind to or react with the deduced binding or reactive site; and

(d) testing whether the candidate compounds are indeed agonists, antagonists or inhibitor.

The present invention also includes a compound obtainable by the above screening method. However, the screening method of the present invention is not limited to the above method. The present invention also includes a process for producing the pharmaceutical composition by the method of above item (14).

There is no special limitation to the above candidate compounds. Such compounds include low molecular weight compounds and peptides. They may be artificially synthesised compounds and naturally occurring compounds. As the compounds obtained by the above screening methods have a function as inhibiting or activating Elk1 phosphorylation action or kinase which phosphorylates Elk1, they are useful as therapeutic or preventive pharmaceuticals for the treatment of diseases resulting from unfavorable activation or inactivation of Elk1 phosphorylation action or kinase which phosphorylates Elk1. In order to isolate and purify the target compounds from the mixture, it is suitable to combine the known methods such as filtration, extraction, washings, drying, concentration, crystallization, various chromatography. When obtainment of a salt of the compounds is desired, a compound which is obtained in the form of a salt can be purified as it is. A compound which is obtained in the free form can be converted into a salt by isolating and purifying a salt obtained by dispersing or dissolving the compound into a suitable solvent and then adding a desired acid or base. Examples of a step to optimize the compounds or salts thereof obtained by the method of the present invention as a pharmaceutical composition, include a step of formulating the compounds or salts thereof according to ordinary processes such as the following. The above compounds or their pharmaceutically acceptable salts in an amount effective as an active ingredient, and pharmaceutically acceptable carriers can be mixed. Further, a form of formulation suitable for the mode of administration is selected. A composition suitable for oral administration includes a solid form such as tablet, granule, capsule, pill and powder, and solution form such as solution, syrup, elixir and dispersion. A form useful for parenteral administration includes sterile solution, dispersion, emulsion and suspension. The above carriers include, for example, sugars such as gelatin, lactose and glucose, starches such corn, wheat, rice and maize, fatty acids such as stearic acid, salts of fatty acids such as calcium stearate, magnesium stearate, talc, vegetable oil, alcohol such as stearyl alcohol and benzyl alcohol, gum, and polyalkylene glycol. Examples of such liquid carriers include generally water, saline, sugar solution of dextrose and the like, glycols such as ethylene glycol, propylene glycol and polyethylene glycol.

The present invention also includes a kit for screening a compound for activity as an inhibitor or activator of Elk1 phosphorylation and/or activation of a kinase which phosphorylates Elk1. The kit comprises reagents, etc. necessary for screening compounds for activity as an inhibitor or activator of Elk1 phosphorylation and/or activation of a kinase which phosphorylates Elk1, including:

(a) a cell comprising a gene encoding a protein that phosphorylates Elk1 and/or activates kinase which phosphorylates Elk1, and a component capable of providing a detectable signal upon phosphorylation of Elk1; and

(b) reagents for measuring the detectable signal.

In another aspect, the present invention relates to a diagnostic kit which comprises:

(a) a polynucleotide of the present invention having a nucleotide sequence shown in SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151 or 153;

(b) a polynucleotide having a nucleotide sequence complementary to the nucleotide sequence of (a);

(c) a protein of the present invention having an amino acid sequence shown in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 or 154 or a fragment thereof; or

(d) an antibody to the protein of the present invention, such of (c) above.

A kit, which comprises at least one of (a), (b), (c) or (d), is useful for diagnosing a disease or susceptibility to a disease such as inflammation, autoimmune diseases, infectious diseases (e.g., HIV infection) and cancers.

Because MAP kinase cascade is involved in a wide variety of pathological conditions such as inflammation, autoimmune diseases, cancers and viral infections, it is an attractive target for drug design and therapeutic intervention. Many experiments show that the inhibition of MAP kinase cascade activity may have significant physiological effects [e.g., J Cell Biochem Apr. 3-27, 2001; 82(1): 68-77, Pharmacol Res March 2001; 43(3): 275-83, Diabetes June 2001; 50(6): 1495-504, Diabetes June 2001; 50(6): 1464-71, Atherosclerosis May 2001; 156 (1): 81-90]. The finding of the new protein described herein capable of phosphorylating Elk1 and/or activating kinase which phosphorylates Elk1 has provided a new method for inhibiting an abnormal MAP kinase cascade activation. Thus, the present invention also relates to use of a compound which inhibits the function of the protein capable of phosphorylating Elk1 and/or activating kinase which phosphorylates Elk1 described above, for inhibiting MAP kinase cascade activation. The compound obtained by the above screening method, which inhibits Elk1 phosphorylation action or kinase which phosphorylates Elk1, is useful as a medicament to treat or prevent diseases characterized by undesirable activation of MAP kinase cascade, such as inflammation, autoimmune diseases, infectious diseases (e.g., HIV infection) and cancers. It is known that there are cases where MAP kinase cascade activation induces apoptosis, and it is thought that there is a possibility that an inhibitor of Elk1 phosphorylation action and/or a kinase which phosphorylates Elk1 will control apoptosis. Diseases which may be treated by the inhibition of apoptosis include GVHD, skin diseases such as toxic epidermal necrolysis (TEN), proliferative nephritides (e.g., IgA nephritis, purpuric nephritis and lupus nephritis) and fulminant hepatitis. In contrast, examples of patients for whom the induction of apoptosis may be exploited in treatment include tumor patients. Thus, the compound obtained by the above screening method, which promotes Elk1 phosphorylation action, is useful as a medicament to treat or prevent these diseases.

In addition, the gene encoding the protein of the present invention is useful for gene therapy to treat various diseases such as cancers, autoimmune diseases, allergy diseases and inflammatory response. “Gene therapy” refers to administering into the human body a gene or a cell into which a gene has been introduced. The protein of the present invention and the DNA encoding the protein can also be used for diagnostic purposes.

The compound obtained by the screening method of the present invention or a salt thereof can be formulated into the above pharmaceutical compositions (e.g., tablets, capsules, elixirs, microcapsules, sterile solutions and suspensions) according to conventional procedures. The formulations thus obtained are safe and of low toxicity, and can be administered, for example, to humans and mammals (e.g., rats, rabbits, sheep, pigs, cattle, cats, dogs and monkeys). Administration to patients can be carried out by methods known in the art, such as intra-arterial injection, intravenous injection and subcutaneous injection. The dosage may vary with the weight and age of the patient as well as a mode of administration, but those skilled in the art can appropriately select suitable dosages. When the compound can be encoded by DNA, the DNA can be inserted into a vector for gene therapy, and gene therapy can be carried out. The dosage and mode of administration may vary with the weight, age and symptoms of the patient, but those skilled in the art can appropriately select them. Thus, the present invention also relates to a pharmaceutical composition which comprises the above compound as an active ingredient.

In addition, the above compound is useful as a medicament to treat or prevent diseases characterized by undesirable activation of MAP kinase cascade, such as inflammation, autoimmune diseases, viral diseases, infectious diseases and cancers. Thus, the present invention also relates to a pharmaceutical composition for inflammation, autoimmune diseases, viral diseases, cancers, etc., which comprises the above compound. Specifically, the pharmaceutical composition is useful as a therapeutic and prophylactic drug against, for example, rheumatoid arthritis, osteoarthritis, systemic lupus erythematosus, diabetes, sepsis, asthma, allergic rhinitis, ischemic heart diseases, inflammatory intestinal diseases, subarachnoid hemorrhage, viral hepatitis, AIDS, GVHD, skin conditions such as toxic epidermal necrolysis (TEN), hyperplastic nephritis (IgA nephritis, purpuric nephritis, lupus nephritis), and fulminant hepatitis.

The present invention also relates to the use of a pharmaceutical composition produced according to above item (14) for manufacturing a medicament against inflammation, autoimmune diseases, viral diseases, cancers, etc. The present invention also includes an antisense oligonucleotide against a gene of any one of above items (3) to (6). An antisense oligonucleotide refers to an oligonucleotide complementary to the target gene sequence. The antisense oligonucleotide can inhibit the expression of the target gene by inhibiting RNA functions such as translation to proteins, transport to the cytoplasm and other activity necessary for overall biological functions. In this case, the antisense oligonucleotide may be RNA or DNA. The DNA sequence of the present invention can be used to produce an antisense oligonucleotide capable of hybridizing with the mRNA transcribed from the gene encoding the protein of the present invention. It is known that an antisense oligonucleotide generally has an inhibitory effect on the expression of the corresponding gene (see e.g., Saibou Kougaku Vol.13, No.4 (1994)). The oligonucleotide containing an antisense coding sequence against a gene encoding the protein of the present invention can be introduced into a cell by standard methods. The oligonucleotide effectively blocks the translation of mRNA of the gene encoding the protein of the present invention, thereby blocking its expression and inhibiting undesirable activity.

The oligonucleotide of the present invention may be a naturally occurring oligonucleotide or its modified form [see e.g., Murakami & Makino, Saibou Kougaku Vol.13, No.4, p.259-266 (1994); Akira Murakami, Tanpakushitsu Kakusan Kouso (PROTEIN, NUCLEIC ACID AND ENZYME) Vol.40, No.10, p.1364-1370 (1995),Tunenari Takeuchi et al., Jikken Igaku (Experimental Medicine) Vol. 14, No. 4 p85-95(1996)]. Thus, the oligonucleotide may have modified sugar moieties or inter-sugar moieties. Examples of such modified forms include phosphothioates and other sulfur-containing species used in the art. According to several preferred embodiments of the present invention, at least one phosphodiester bond in the oligonucleotide is substituted with the structure which can enhance the ability of the composition to permeate cellular regions where RNA with the activity to be regulated is located.

Such substitution preferably involves a phosphorothioate bond, a phosphoramidate bond, methylphosphonate bond, or a short-chain alkyl or cycloalkyl structure. The oligonucleotide may also contain at least some modified base forms. Thus, it may contain purine and pyrimidine derivatives other than naturally occurring purine and pyrimidine. Similarly, the furanosyl moieties of the nucleotide subunits can be modified so long as the essential purpose of the present invention is attained. Examples of such modifications include 2′—O-alkyl and 2′-halogen substituted nucleotides. Examples of modifications in sugar moieties at their 2-position include OH, SH, SCH₃, OCH₃, OCN or O(CH₂)ₙCH₃, wherein n is 1 to about 10, and other substituents having similar properties. All the analogues are included in the scope of the present invention so long as they can hybridize with the mRNA of the gene of the present invention to inhibit functions of the mRNA.

The oligonucleotide of the present invention contains about 3 to about 50 nucleotides, preferably about 8 to about 25 nucleotides, more preferably about 12 to about 20 nucleotides. The oligonucleotide of the present invention can be produced by the well-known solid phase synthesis technique. Devices for such synthesis are commercially available from some manufactures including Applied Biosystems. Other oligonucleotides such as phosphothioates can also be produced by methods known in the art.

The oligonucleotide of the present invention is designed to hybridize with the mRNA transcribed from the gene of the present invention. Those skilled in the art can easily design an antisense oligonucleotides based on a given gene sequence (For example, Murakami and Makino: Saibou Kougaku Vol. 13 No.4 p259-266 (1994), Akira Murakami: Tanpakushitsu Kakusan Kouso (PROTEIN, NUCLEIC ACID AND ENZYME) Vol. 40 No.10 p1364-1370 (1995), Tunenari Takeuchi et al., Jikken Igaku (Experimental Medicine) Vol. 14 No. 4 p85-95 (1996)). Recent study suggests that antisense oligonucleotides which are designed in a region containing 5′ region of mRNA, preferably, the translation initiation site, are most effective for the inhibition of the expression of a gene. The length of the antisense oligonucleotides is preferably 15 to 30 nucleotides and more preferably 20 to 25 nucleotides. It is important to confirm no interaction with other mRNA and no formation of secondary structure in the oligonucleotide sequence by homology search. The evaluation of whether the designed antisense oligonucleotide is functional or not can be determined by introducing the antisense oligonucleotide into a suitable cell and measuring the amount of the target mRNA, for example by northern blotting or RT-PCR, or the amount of the target protein, for example by western blotting or fluorescent antibody technique, to confirm the effect of expression inhibition.

Another method includes the triple helix technique. This technique involves forming a triple helix on the targeted intra-nuclear DNA sequence, thereby regulating its gene expression, mainly at the transcription stage. The oligonucleotide is designed mainly in the gene region involved in the transcription and inhibits the transcription and the production of the protein of the present invention. Such RNA, DNA and oligonucleotide can be produced using known synthesizers.

The oligonucleotide may be introduced into the cells containing the target nucleic acid sequence by any of DNA transfection methods such as calcium phosphate method, electroporation, lipofection, microinjection, or gene transfer methods including the use of gene transfer vectors such as viruses. An antisense oligonucleotide expression vector can be prepared using a suitable retrovirus vector, then the expression vector can be introduced into the cells containing the target nucleic acid sequence by contacting the vector with the cells in vivo or ex vivo.

The DNA of the present invention can be used in the antisense RNA/DNA technique or the triple helix technique for the purpose of inhibiting MAP kinase cascade activation mediated by the protein of the present invention.

The antisense oligonucleotide against the gene encoding the protein of the present invention is useful as a medicament to treat or prevent diseases characterized by undesirable activation of MAP kinase cascade, such as inflammation, autoimmune diseases, infectious diseases (e.g., HIV infection) and cancers. Thus, the present invention also includes a pharmaceutical composition which comprises the above antisense oligonucleotide as an active ingredient. The antisense oligonucleotide can also be used to detect such diseases using northern hybridization or PCR.

The present invention also includes a ribozyme which inhibits Elk1 phosphorylation action and/or kinase which phosphorylates Elk1. A ribozyme is an RNA capable of recognizing a nucleotide sequence of a nucleic acid and cleaving the nucleic acid (see e.g., Hiroshi Yanagawa, “Jikken Igaku (Experimental Medicine) Bioscience 12: New Age of RNA). The ribozyme can be produced so that it cleaves the selected target RNA (e.g., mRNA encoding the protein of the present invention). Based on the nucleotide sequence of the DNA encoding the protein of the present invention, the ribozyme specifically cleaving the mRNA of the protein of the present invention can be designed. Such ribozyme has a complementary sequence to the mRNA for the protein of the present invention, complementarily associates with the mRNA and then cleaves the mRNA, which results in reduction or entire loss of the expression of the protein of the present invention. The level of the reduction of the expression is dependent on the level of the ribozyme expression in the target cells.

There are two types of ribozyme commonly used: a hammerhead ribozyme and a hairpin ribozyme. In particular, hammerhead ribozymes have been well studied regarding their primary and secondary structure necessary for their cleavage activity, and those skilled in the art can easily design the ribozyme nucleotides solely on the nucleotide sequence information for the DNA encoding the protein of the present invention [see e.g., Iida et al., Saibou Kougaku Vol.16, No.3, p.438-445 (1997); Ohkawa & Taira, Jikken Igaku (Experimental Medicine) Vol.12, No.12, p.83-88 (1994)]. It is known that the hammerhead ribozymes have a structure consisting of two recognition sites (recognition site I and recognition site II forming a chain complementary to target RNA) and an active site, and cleave the target RNA at the 3′ end of its sequence NUX (wherein N is A or G or C or U, and X is A or C or U)after the formation of a complementary pair with the target RNA in the recognition sites. In particular, the sequence GUC (or GUA) has been found to have the highest activity [see e.g., Koizumi, M. et al., Nucl. Acids Res. 17:7059-7071 (1989); Iida et al., Saibou Kougaku Vol.16, No.3, p.438-445 (1997); Ohkawa & Taira, Jikken Igaku (Experimental Medicine) Vol.12, No.12, p.83-88 (1994); Kawasaki & Taira, Jikken Igaku (Experimental Medicine) Vol.18, No.3, p.381-386 (2000)].

Therefore the sequence GTC (or GTA) is searched out, and a ribozyme is designed to form several, up to 10 to 20 complementary base pairs around that sequence. The suitability of the designed ribozyme can be evaluated by checking whether the prepared ribozyme can cleave the target mRNA in vitro according to the method described for example in Ohkawa & Taira, Jikken Igaku (Experimental Medicine) Vol.12, No.12, p.83-88 (1994). The ribozyme can be prepared by methods known in the art to synthesize RNA molecules.

Alternatively, the sequence of the ribozyme can be synthesized on a DNA synthesizer and inserted into various vectors containing a suitable RNA polymerase promoter (e.g., T7 or SP6) to enzymatically synthesize an RNA molecule in vitro. Such ribozymes can be introduced into cells by gene transfer methods such as microinjection. Another method involves inserting a ribozyme DNA into a suitable expression vector and introducing the vector into cell strains, cells or tissues. Suitable vectors can be used to introduce the ribozyme into a selected cell. Examples of vectors commonly used for such purpose include plasmid vectors and animal virus vectors (e.g., retrovirus, adenovirus, herpes or vaccinia virus vectors). Such ribozymes are capable of inhibiting the Elk1 phosphorylation action and/or kinase which phosphorylates Elk1 mediated by the protein of the present invention.

As mentioned above, full-length cDNA is used in the present invention. That means 5′ end sequence of the cDNA in the present invention is the transcription initiation site of the corresponding mRNA. Therefore the cDNA sequence can be used to identify the promoter region of the gene by comparing the cDNA with the genomic nucleotide sequence. Genomic nucleotide sequences are available from various databases when the sequences have been deposited in the databases. Alternatively, the cDNA can also be used to clone the desired sequence from a genomic library, for example, by hybridization, and determine its nucleotide sequence. Thus, by comparing the nucleotide sequence of the cDNA of the present invention with a genomic sequence, the promoter region of the gene located upstream the cDNA can be identified. In addition, the promoter fragment thus identified can be used to construct a reporter plasmid for evaluating the expression of the gene. In general, the DNA fragment spanning 2 kb (preferably 1 kb) upstream from the transcription initiation site can be inserted upstream of the reporter gene to produce the reporter plasmid. The reporter plasmid can be used to screen for a compound which enhances or reduces the expression of the gene. For example, such screening can be carried out by transforming a suitable cell with the reporter plasmid, culturing the transformed cell for a certain period of time, adding a certain amount of a test compound, measuring the reporter activity expressed by the cell after a certain period of time, and comparing the activity with that of a cell to which the test compound has not been added. These methods are also included in the scope of the present invention.

The present invention also relates to a computer-readable medium on which a sequence data set has been stored, said sequence data set comprising at least one nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135 137, 139, 141, 143, 145, 147, 149, 151 and 153 and/or at least one amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 and 154.

In another aspect, the present invention relates to a method for calculating a homology, which comprises comparing data on the above medium with data of other nucleotide sequences. Thus, the gene and amino acid sequence of the present invention provide valuable information for determining their secondary and tertiary structure, e.g., information for identifying other sequence having a similar function and high homology. These sequences are stored on the computer-readable medium, then a database is searched using data stored in a known macromolecule structure program and a known search tool such as GCG program package (Devereux, J. et al, Nucleic Acids Research 12(1):387 (1984)). In this manner, a sequence in a database having a certain homology can be easily found.

The computer-readable medium may be any composition of materials used to store information or data. Examples of such media include commercially available floppy disks, tapes, chips, hard discs, compact disks and video disks. The data on the medium allows a method for calculating a homology by comparing the data with other nucleotide sequence data. This method comprises the steps of providing a first polynucleotide sequence containing the polynucleotide sequence of the present invention for the computer-readable medium, and then comparing the first polynucleotide sequence with at least one second polynucleotide or polypeptide sequence to identify the homology.

The present invention also relates to an insoluble substrate to which a polynucleotide comprising all or part of a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151 and 153, is fixed. A plurality of the various polynucleotides which are DNA probes are fixed on a specifically processed solid substrates such as slide glass to form a DNA microarray and then a labeled target polynucleotide is hybridized with the fixed polynucleotides to detect a signal from each of the probes. The data obtained is analyzed and the gene expression is determined.

The present invention further relates to an insoluble substrate to which a polypeptide comprising all or part of an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 and 154 is fixed. Proteins, which are expected to be useful for diagnosis or development of a new drug, can be isolated and/or identified by mixing the insoluble substrate and a cell extract from organisms, and capturing the proteins onto the insoluble substrate.

### EXAMPLES

The following examples further illustrate, but do not limit the present invention.

### Example 1

### Construction of a Full-Length cDNA Library Using the Oligo-Capping Method

(1) Preparation of RNA from Human Lung Fibroblasts (Cryo NHLF)

Human lung fibroblasts (Cryo NHLF: purchased from Sanko Junyaku Co., Ltd.) were cultured according to the attached protocol. After the cells were cultured to obtain fifty 10 cm dishes, the cells were recovered with a cell scraper. Then, total RNA was obtained from the recovered cells by using the RNA extraction reagent ISOGEN (purchased from NIPPON GENE) according to the manufacture's protocol. Then, poly A⁺ RNA was obtained from the total RNA by using an oligo-dT cellulose column according to Maniatis et al., supra.

(2) Preparation of RNA from Mouse ATDC5 Cells

ATDC5, a cell strain cloned from mouse EC (embryonal carcinoma) (Atsumi, T. et al.: Cell Diff. Dev., 30: p109-116)(1990) was repeatedly subcultured to obtain fifty 10 cm dishes containing the resultant culture. Thereafter, poly A⁺ RNA was obtained by a method similar to that of (1) above. Culture of ATDC5 cells was performed according to the method described in Atsumi, T. et al.: Cell Diff. Dev., 30: p109-116 (1990).

(3) Construction of a Full-Length cDNA Library by the Oligo-Capping Method

A full-length cDNA library was constructed from the above poly A⁺ RNA by the oligo-capping method according to the method of Sugano S. et al. [e.g., Maruyama, K. & Sugano, S., Gene, 138:171-174 (1994); Suzuki, Y. et al., Gene, 200:149-156 (1997); Suzuki, Y. & Sugano, S. “Shin Idenshi Kougaku Handbook (New Genetic Engineering Handbook)”, the third edition (1999), an extra issue of “Jikken Igaku (Experimental Medicine)”, YODOSHA CO., LTD.].

(4) Preparation of Plasmid DNA

The full-length cDNA library constructed as above was transfected into E. coli strain TOP 10 by electroporation, then spread on LB agar medium, and incubated overnight at 37° C. Then, using QIAwell 96 Ultra Plasmid Kit (QIAGEN) according to the manufacturer's protocol, the plasmids were recovered from the colonies grown on ampicillin-containing LB agar medium.

### Example 2

### Cloning of DNA Capable of Phosphorylating Elk1 and/or Activating Kinase which Phosphorylates Elk1

(1) Screening of the cDNA Encoding the Protein Capable of Phosphorylating Elk1 and/or Kinase which Phosphorylates Elk1

293-EBNA cells (purchased from Invitrogen) were grown to 1×10⁴ cells/well in a 96 well plate for cell culture for 24 hours at 37° C. (in the presence of 5% CO₂) using 5% FBS containing DMEM medium. Then, 60 ng of pFR-Luc (purchased from STRATAGENE), 0.25 ng of pFA2-Elk1 (purchased from STRATAGENE), 5 ng of pcDNA3.1 (+) (INVITROGEN) into which human p38 had been incorporated, 30 ng of pcDNA3.1(+) into which human JNJ 1 β 1 gene had been incorporated; and 2 μl of the full-length cDNA prepared in above Example 1.(3) were cotransfected into the cells in a well using FuGENE 6 (purchased from Roche) according to the manufacturer's protocol. After 24 hours of culture at 37° C., the reporter activity reflecting activation of MAP kinase cascade (luciferase activity) was measured using long-term luciferase assay system, PIKKA GENE LT2.0 (TOYO INK) according to the attached manufacturer's instructions. The luciferase activity was measured using Wallac ARVO™ ST 1420 MULTILABEL COUNTER (Perkin Elmer).

(2) DNA Sequencing

The above screening was carried out for 165,000 clones, and plasmids showing a 5-fold or more increase in luciferase activity compared to that of the control experiment (luciferase activity of the cell into which vacant vector pME18S-FL3 is introduced instead of full-length cDNA) were selected. One pass sequencing was carried out from the 5′ end of the cloned cDNA (sequencing primer: 5′-CTTCTGCTCTAAAAGCTGCG-3′ (SEQ ID NO: 155)) and from the 3′ end (sequencing primer: 5′-CGACCTGCAGCTCGAGCACA-3′ (SEQ ID NO: 156)) so that as long sequence as possible is determined. The sequencing was carried out using the reagent Thermo Sequenase II Dye Terminator Cycle Sequencing Kit (Amersham Pharmacia Biotech) or BigDye Terminator Cycle Sequencing FS Ready Reaction Kit (Applied Biosystems) and the device ABI PRISM 377 sequencer or ABI PRISM 3100 sequencer according to the manufacturer's instructions.

(3) Database Analysis of the Obtained Clones

BLAST (Basic local alignment search tool) searching [S. F. Altschul et al., J. Mol. Biol., 215:403-410 (1990)] was carried out in GenBank for the obtained nucleotide sequences. The results showed that 70 clones represented 37 genes encoding new proteins capable of phosphorylating Elk1 and/or kinase which phosphorylates Elk1.

(4) Full-Length Sequencing

The full-length DNA sequences for the 37 new clones were determined (SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151 and 153). The amino acid sequences of the protein coding regions (open reading frames) were deduced (SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 and 154).

### Example 3

### Screening Compounds Inhibiting Activation of MAP Kinase Cascade, in Particular, p38 Signal

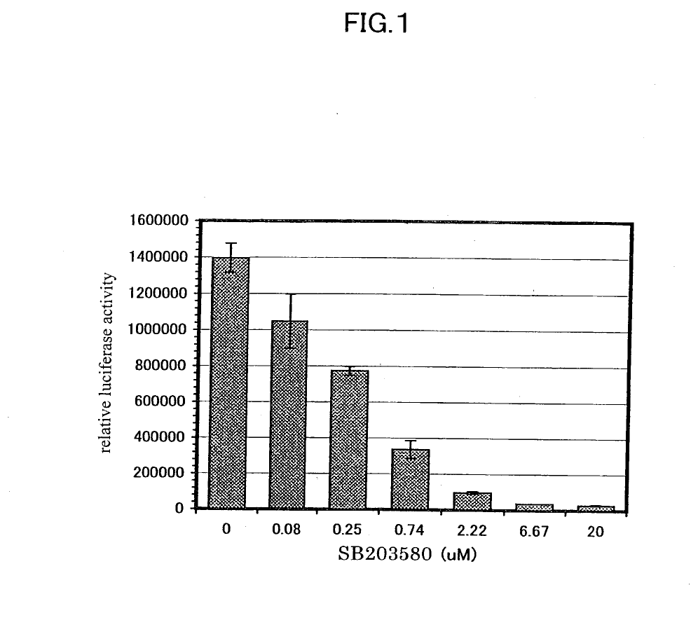

293-EBNA cells were seeded on DMEM medium containing 5% FBS in a 96-well cell culture plate to a final cell density of 1×10⁴ cells/100 μl medium/well, and cultured for 24 hours at 37° C. in the presence of 5% CO₂. Then, 10 ng of the plasmid containing the gene encoding MKK3, a protein known to act to activate MAP kinase cascade, obtained by the screening in Example 2 and 60 ng of pFR-Luc (purchased from STRATAGENE), 0.25 ng pFA2-Elk1 (purchased from STRATAGENE), 5 ng of pcDNA3.1 (+) (INVITROGEN) into which human p38 had been incorporated were cotransfected into the cells in a well using FuGENE 6. Within 1 hour, SB203580 (purchased from CALBIOCHEM) which is known to be an inhibitor of p38, which is one kind of MAP kinase, was added to the culture to a final concentration of 0.082-20 μM. After 24 hours of culture at 37° C., the reporter activity was measured using PIKKA GENE LT2.0. The results showed that SB203580 inhibited the expression of the reporter gene to a level of IC50 is about 0.4 μM (FIG. 1).

### Example 4

### Measurement of Reporter Activity Corrected using an Internal Control

293-EBNA cells (purchased from INVITROGEN) were inoculated on a 5% FBS (Fetal Bovine Serum)-containing DMEM medium in a 96-well cell culture plate to a final cell density of 1×10⁴ cells/well, and cultured for 24 hours at 37° C. (in the presence of 5% CO₂). Then, using FuGENE 6 (purchased from Roche), 12.5 ng, 25 ng, 50 ng and 100 ng of each of the expression plasmids obtained in Example 2 above which comprise a gene encoding the MAP kinase cascade activating protein according to SEQ ID NO: 4, 6, 12, 16, 18, 22, 26, 28, 30, 44, 50, 52, 60, 68, 72, 104, 110, 112, 116, 122, 132, 142, 146, 148, 150, 152 or 154 were respectively co-transfected in a well with 60 ng of pFR-Luc (purchased from STRATAGENE), 0.25 ng of pFA2-Elk1 (STRATAGENE), 5 ng of pcDNA 3.1 (+) (INVITROGEN) into which human p38 gene had been incorporated, 30 ng of pcDNA 3.1 (+) (INVITROGEN) into which human JNJ 1 β 1 gene had been incorporated, and 10 ng of pRL-tk (purchased from Toyo Ink) as a reporter gene for internal control use. The method used for introduction was in accordance with the attached protocol. After culturing for 24 hours at 37° C., reporter activity (firefly luciferase acitivity), which reflects MAP kinase cascade activation, and reporter activity being the internal control (sea pansy luciferase) were measured using Picagene Dual (purchased from Toyo Ink Manufacturing) in accordance with the manufacturer's instructions.

Luciferase activity was measured with Perkin Elmer's Wallac ARVOTMST 1420 MULTILABEL COUNTER.

As a result, reporter activity, which reflects MAP kinase cascade activation corrected with internal control reporter activity, exhibited increases that were dependent on the amount of expression plasmid comprising a gene encoding a MAP kinase cascade activating protein according to SEQ ID NO: 4, 6, 12, 16, 18, 22, 26, 28, 30, 44, 50, 52, 60, 68, 72, 104, 110, 112, 116, 122, 132, 142, 146, 148, 150, 152 or 154 obtained in Example 2 above, as the amount of plasmid introduced was changed through 12.5 ng, 25 ng, 50 ng, and 100 ng, respectively.

Reporter activity where an expression plasmid comprising the above gene was introduced, which reflects MAP kinase cascade activation corrected with internal control reporter activity, is shown in Table 1, which is expressed as a factor of the same reporter activity where a null vector was introduced (S/B value).

TABLE 1

| SEQ ID NO: | 12.5 ng/well | 25 ng/well | 50 ng/well | 100 ng/well |
|---|---|---|---|---|
| 26 | ND | 1.6 | 3.2 | 4.1 |
| 12 | ND | 2.1 | 2.7 | 3.9 |
| 4 | ND | 2.1 | 4.1 | 4.5 |
| 28 | ND | 1.8 | 2.3 | 2.9 |
| 30 | ND | 4.2 | 6.3 | 7.6 |
| 50 | ND | 2.3 | 4.3 | 3.8 |
| 142 | ND | 5.3 | 6.6 | 5.8 |
| 68 | ND | 2.3 | 4.0 | 7.3 |
| 60 | ND | 2.0 | 2.6 | 4.2 |
| 52 | ND | 2.3 | 3.6 | 5.4 |
| 146 | ND | 3.5 | 5.6 | 8.8 |
| 148 | ND | 1.6 | 3.4 | 5.0 |
| 72 | ND | 2.2 | 5.9 | 7.7 |
| 44 | ND | 9.1 | 55.4 | 64.5 |
| 22 | ND | 24.2 | 50.8 | 69.3 |
| 16 | ND | 3.4 | 8.4 | 8.1 |
| 6 | ND | 2.1 | 2.4 | 2.9 |
| 18 | ND | 1.8 | 2.3 | 2.9 |
| 104 | 3.8 | 7.0 | 14.7 | ND |
| 110 | 2.3 | 3.4 | 4.7 | ND |
| 116 | 11.9 | 17.3 | 19.9 | ND |
| 122 | 1.8 | 2.9 | 6.8 | ND |
| 132 | 5.6 | 10.1 | 16.6 | ND |
| 112 | 7.6 | 2.2 | 3.1 | ND |
| 150 | 6.7 | 2.8 | 1.7 | ND |

ND: No data

### Example 5

### Screening for Inhibitors

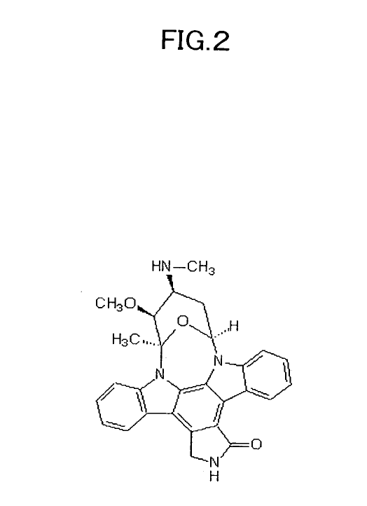

293-EBNA cells (INVITROGEN) were inoculated on a 5% FBS (Fetal Bovine Serum)-containing DMEM medium in a 96-well cell culture plate to a final cell density of 1×10⁴ cells/well, and cultured for 24 hours at 37° C. (in the presence of 5% CO₂). Then, using FuGENE6 (Roche), 50 ng of each of the expression plasmids obtained in Example 2 above which comprise a gene encoding the MAP kinase cascade activating protein according to SEQ ID NO: 4, 6, 12, 16, 18, 22, 26, 28, 30, 44, 50, 52, 60, 68, 72, 104, 110, 112, 116, 122, 132, 142, 146, 148, 150, 152 or 154 were respectively co-transfected in a well with 60 ng of pFR-Luc (STRATAGENE), 0.25 ng of pFA2-Elk1 (STRATAGENE), 5 ng of pcDNA 3.1 (+) (INVITROGEN) into which human p38 gene had been incorporated and 30 ng of pcDNA 3.1 (+) (INVITROGEN) into which human JNJ1 β 1 gene had been incorporated. The method used for introduction was in accordance with the attached protocol. After culturing for 2 hours at 37° C., any one of 1000 types of compound that were held in stock, was added to a final concentration of 10 μM per well, and after further culturing for 24 hours at 37° C., reporter activity, which reflects MAP kinase cascade activition, was measured by Picagene LT2 (purchased from Toyo Ink Manufacturing) in accordance with the manufacturer's instructions. Using a well to which no compound was added as a control, compounds were screened to identify a compound which reduced reporter activity in the well to which it was added to 50% or lower. As a result, in systems to which was introduced an expression plasmid comprising a gene encoding the MAP kinase cascade activating protein according to SEQ ID NO: 4, 6, 12, 16, 18, 22, 26, 28, 30, 44, 50, 52, 60, 68, 72, 104, 110, 112, 116, 122, 132, 142, 146, 148, 150, 152 or 154, staurosporine, which is known to inhibit the activity of various kinases non-specifically, was obtained as an inhibitor. The structural formula of the staurosporine obtained here is shown in FIG. 2.

### EFFECTS OF THE INVENTION

As described above, the present invention provides proteins capable of phosphorylating Elk1 and/or activating a kinase which phosphorylates Elk1, and genes encoding the proteins. These proteins and genes are highly likely to posses highly industrially useful MAP kinase cascade activating activity. The proteins of the present invention and the genes encoding the proteins allow not only screening for compounds useful for treating and preventing diseases associated with the excessive activation or inhibition of MAP kinase cascade, but also production of diagnostics for such diseases. The genes of the present invention are also useful as a gene source used for gene therapy.

1. A purified protein selected from the group consisting of:
(a) a protein which consists of an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 and 154; and
(b) a protein that acts to phosphorylate Elk1 and/or activate a kinase which phosphorylates Elk1, and consists of an amino acid sequence having at least one amino acid deletion, substitution or addition in an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 and 154.

2. A purified protein that acts to phosphorylate Elk1 and/or activate a kinase which phosphorylates Elk1, and comprises an amino acid sequence having at least 95% identity to the protein according to claim 1 over the entire length thereof.

3. An isolated polynucleotide which comprises a nucleotide sequence encoding a protein selected from the group consisting of:
(a) a protein which comprises an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 and 154; and
(b) a protein that acts to phosphorylate Elk1 and/or activate a kinase which phosphorylates Elk1, and consists of an amino acid sequence having at least one amino acid deletion, substitution or addition in an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 and 154.

4. An isolated polynucleotide comprising a polynucleotide sequence selected from the group consisting of:
(a) a polynucleotide sequence represented by SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151 and 153; and a polynucleotide sequence complementary to said polynucleotide sequence;
(b) a polynucleotide sequence which encodes a protein that acts to phosphorylate Elk1 and/or activate a kinase which phosphorylates Elk1, and which hybridizes under stringent conditions with a polynucleotide having a polynucleotide sequence that is complementary to the polynucleotide sequence of (a);
(c) a polynucleotide sequence encoding a protein that acts to phosphorylate Elk1 and/or activate a kinase which phosphorylates Elk1, and consists of a nucleotide sequence having at least one nucleotide deletion, substitution or addition in a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151 and 153.

5. An isolated polynucleotide comprising a nucleotide sequence which encodes a protein that acts to phosphorylate Elk1 and/or activate a kinase which phosphorylates Elk1, and has at least 95% identity to the polynucleotide sequence according to claim 3 over the entire length thereof.

6. An isolated polynucleotide comprising a nucleotide sequence which encodes a protein that acts to phosphorylate Elk1 and/or activate a kinase which phosphorylates Elk1, and has at least 95% identity to the polynucleotide sequence according to claim 4 over the entire length thereof.

7. A purified protein encoded by the polynucleotide according to any one of claims 3 to 6.

8. A recombinant vector which comprises a polynucleotide according to any one of claims 3 to 6.

9. A transformed cell which comprises the recombinant vector according to claim 8.

10. A membrane of the cell according to claim 9, when the protein according to claim 1, 2 or 7 is a membrane protein.

11. A process for producing a protein comprising:
(a) culturing a transformed cell comprising the isolated polynucleotide according to any one of claims 3 to 6 under conditions providing expression of the encoded protein; and
(b) recovering the protein from the culture.

12. A process for diagnosing a disease or susceptibility to a disease in a subject related to expression or activity of the protein of claim 1, 2 or 7 in the subject, comprising:
(a) determining the presence or absence of a mutation in the nucleotide sequence encoding said protein in the genome of said subject; and/or
(b) analyzing the amount of expression of said protein in a sample derived from said subject.

13. A method for screening compounds for inhibiting or promoting activity for Elk1 phosphorylation action and/or a kinase which phosphorylates Elk1, which comprises the steps of:
(a) providing a cell with a gene encoding a protein that acts to phosphorylate Elk1 and/or activate a kinase which phosphorylates Elk1, and a component that provides a detectable signal associated with Elk1 phosphorylation action and/or a kinase which phosphorylates Elk1;
(b) culturing a transformed cell under conditions, which permit the expression of the gene in the transformed cell;
(c) contacting the transformed cell with one or more compounds;
(d) measuring the detectable signal; and
(e) isolating or identifying a compound as an activator compound or an inhibitor compound according to measurement of the detectable signal.

14. A process for producing a pharmaceutical composition, which comprises the steps of:
(a) providing a cell with a gene encoding a protein that acts to phosphorylate Elk1 and/or activate a kinase which phosphorylates Elk1, and a component capable of providing a detectable signal;
(b) culturing a transformed cell under conditions, which permit the expression of the gene in the transformed cell;
(c) contacting the transformed cell with one or more compounds;
(d) measuring the detectable signal;
(e) isolating or identifying a compound as an activator compound or an inhibitor compound according to measurement of the detectable signal; and
(f) optimizing the isolated or identified compound for a pharmaceutical composition.

15. A kit for screening a compound for inhibiting or promoting activity for Elk1 phosphorylation action and/or a kinase which phosphorylates Elk1, which comprises:
(a) a cell transformed with a gene encoding a protein that acts to phosphorylate Elk1 and/or activate a kinase which phosphorylates Elk1, and a component that provides a detectable signal upon activation of Elk1 phosphorylation action and/or a kinase which phosphorylates Elk1; and
(b) reagents for measuring the detectable signal.

16. A monoclonal or polyclonal antibody that reacts with the protein according to claim 1, 2 or 7.

17. A process for producing a monoclonal or polyclonal antibody that reacts with the protein of claim 1, 2 or 7, which comprises administering the protein according to claim 1, 2 or 7 or epitope-bearing fragments thereof to a non-human animal.

18. An antisense oligonucleotide complementary to at least a part of the polynucleotide according to any one of claims 3 to 6, which prevents expression of a protein which phosphorylates Elk1 and/or activates a kinase which phosphorylates Elk1.

19. A ribozyme which inhibits activation of Elk1 phosphorylation action and/or a kinase which phosphorylates Elk1 by cleavage of RNA that encodes the protein of claim 1, 2 or 7.

20. A method for treating a disease, which comprises administering to a subject an effective amount of a compound screened by the process according to claim 13, and/or a monoclonal or polyclonal antibody according to clam 16, and/or an antisense oligonucleotide according to claim 18 and/or a ribozyme according to claim 19 for treating a disease selected from the group consisting of inflammation, autoimmune diseases, cancers, viral infections, GVHD, skin diseases, IgA nephritis, purpuric nephritis, proliferative nephritides, and fulminant hepatitis.

21. A pharmaceutical composition produced according to claim 14 for inhibiting or promoting Elk1 phosphorylation action and/or a kinase which phosphorylates Elk1.

22. A pharmaceutical composition according to claim 21 for the treatment of inflammation, autoimmune diseases, cancers, viral infections, GVHD, skin diseases, IgA nephritis, purpuric nephritis, proliferative nephritides, and/or fulminant hepatitis.

23. A method of treating inflammation, autoimmune diseases, cancers, viral infections, GVHD, skin diseases, IgA nephritis, purpuric nephritis, proliferative nephritides, and/or fulminant hepatitis, which comprising administering a pharmaceutical composition produced according to claim 14 to a patient suffering from a disease relating to abnormalities in Elk1 phosphorylation action and/or activation of kinase which phosphorylates Elk1.

24. A pharmaceutical composition which comprises a monoclonal or polyclonal antibody according to claim 16 as an active ingredient.

25. A pharmaceutical composition which comprises an antisense oligonucleotide according to claim 18 as an active ingredient.

26. A pharmaceutical composition according to claim 24 or 25, wherein a target disease is selected from the group consisting of inflammation, autoimmune diseases, cancers, viral infections, GVHD, skin diseases, IgA nephritis, purpuric nephritis, proliferative nephritides, and fulminant hepatitis.

27. A computer-readable medium on which a sequence data set has been stored, said sequence data set comprising at least one nucleotide sequence selected from the group, consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151 and 153; and/or at least one amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 and 154.

28. A method for calculating identity to other nucleotide sequences and/or amino acid sequences, which comprises comparing data on the medium according to claim 27 with data of said other nucleotide sequences and/or amino acid sequences.

29. An insoluble substrate to which polynucleotide comprising all or part of the nucleotide sequences selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151 and 153 are fixed.

30. An insoluble substrate to which polypeptides comprising all or a part of the amino acid sequences selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152 and 154 are fixed.
